(19) United States
(12) Patent Application Publication
(10) Pub. No.: US 2010/0055712 A1
(43) Pub. Date: Mar. 4, 2010

(54) Oligopeptides for treatment of osteoporosis and other bone diseases and methods therefor

(75) Inventor: Guojun Bu, Chesterfield, MO (US)

Correspondence Address: WASHINGTON UNIVERSITY-SNR;C/O SONNENSCHEIN NATH & ROSENTHAL L.L.P, P.O. BOX 061080, WACKER DRIVE STATION , WILLIS TOWER, CHICAGO, IL, 60606, US

(73) Assignee: Washington University, St. Louis, MO (US)

(21) Appl. No.: 12/459,060

(22) Filed: Jun. 26, 2009

Related U.S. Application Data
(60) Provisional application No. 61/133,289, filed on Jun. 27, 2008.

(51) Int. Cl. G01N 33/53

(52) U.S. Cl. 435/7.1

(57) ABSTRACT

Methods of identifying compounds for treating Alzheimer's disease are disclosed. These methods comprise a) forming an in vitro mixture comprising i) cells expressing LDL receptor related protein 1 (LRP1), ii) an LRP1 ligand comprising a label, and iii) a candidate compound; and b) determining quantity of the label incorporated by the cells, whereby a candidate compound is deemed effective for treating Alzheimer's disease if the quantity of label incorporated by the cells exceeds that of a control in vitro mixture comprising cells expressing LRP1 and the LRP1 ligand, but not comprising the compound.

### STATEMENT REGARDING FEDERALLY SPONSORED RESEARCH OR DEVELOPMENT

This invention was made with government support under Grant R01 AG027924 awarded by the National Institutes of Health. The Government has certain rights in the invention.

### INCORPORATION-BY-REFERENCE OF A SEQUENCE LISTING SUBMITTED IN COMPUTER-READABLE FORM

The Sequence Listing, which is a part of the present disclosure, includes a computer readable form and a written sequence listing comprising nucleotide and/or amino acid sequences. The sequence listing information recorded in computer readable form is identical to the written sequence listing. The subject matter of the Sequence Listing is incorporated herein by reference in its entirety.

### FIELD

The present teachings generally relate to LDL-receptor-related protein 1 (LRP1) expression as it relates to Alzheimer's disease and other neurological diseases. The present teachings further relate to screening methods to identify compounds that interact with LRP1 and which can be used for the treatment of Alzheimer's disease.

### BACKGROUND

Mounting genetic and biochemical evidence strongly supports the hypothesis that amyloid β-peptide (Aβ) accumulation in the brain is an early and toxic event in the pathogenesis of Alzheimer's disease (AD) (Hardy and Selkoe, Science 297, 353-356, 2002). Accordingly, reducing brain Aβ production and/or increasing its clearance have become attractive targets for AD drug development (Hardy and Selkoe, 2002). Aβ is derived from sequential proteolytic processing of amyloid precursor protein (APP), a ubiquitously expressed type I transmembrane protein that undergoes two distinct processing pathways (Selkoe and Kopan, Annu. Rev. Neurosci. 26, 565-597, 2003; Zheng and Koo, Molecular Neurodegeneration 1, 5, 2006). In the non-amyloidogenic pathway, APP undergoes ectodomain shedding by α-secretase, identified as members of the ADAM metalloprotease family (Zheng and Koo, 2006). Subsequent cleavage of the APP C-terminal membrane-associated stub by γ-secretase (Selkoe and Kopan, 2003) generates a non-toxic p3 peptide, as well as the APP intracellular domain (AICD) (Selkoe and Kopan, 2003). In the amyloidogenic pathway, APP is first cleaved by the β-secretase β-site APP cleaving enzyme 1 (BACE1) (Vassar et al., Science 286, 735-741, 1999) and then by γ-secretase to generate Aβ and the APP intra-cellular domain (AICD). Mutations associated with early-onset familial forms of AD (FAD) are found in the APP gene itself or in the genes of presenilin 1 (PS1) and PS2, whose products, along with nicastrin, Pen-2, and Aph-1, are obligate components of a multi-protein complex that gives rise to γ-secretase activity (Selkoe and Kopan, 2003). A common feature of all FAD mutations is that they increase the generation of Aβ peptides or the proportion of the longer Aβ42 form, considered to be more amyloidogenic and pathogenic than the shorter Aβ40 due to its highly aggregative nature (Hardy and Selkoe, 2002).

Several biological functions for APP and its processing products have been described (Zheng and Koo, 2006). The notion that APP is a cell surface receptor has long been speculated but remains unproven due to the lack of a bona fide ligand. The function of APP is further complicated by the presence of two APP-related genes, APLP1 and APLP2 (Zheng and Koo, 2006). Deletion of APLP2 and either APP or APLP1 results in early postnatal lethality (Zheng and Koo, 2006), suggesting redundancy between APLP2 and the other two family members. APP ectodomain has been shown to participate in cell adhesion, neurite outgrowth, and synaptogenesis (Zheng and Koo, 2006). AICD, highlighted with an γxNPxY motif for binding of an array of interacting proteins, modulates cell migration, axonal transport, and cell signaling (Zheng and Koo, 2006). The most relevant interacting protein is Fe65, which modulates APP processing and AICD nuclear translocation (Cao et al., Science 293, 115-120, 2001). Knockout of Fe65 and its homolog Fe65L1 results in cortical dysplasia and compromised integrity of the pial basement membrane (Guenette et al., EMBO J. 25, 420-431, 2006). Intriguingly, this phenotype closely resembles that seen in triple-mutant mice lacking the APP family members APP, APLP1, and APLP2 (Guenette et al., 2006; Herms et al., EMBO J. 23, 4106-4115, 2004), strongly suggesting a common signaling pathway that requires the function of both Fe65 and APP. Mice lacking both APP and APLP2 show defective neuromuscular synapses (Wang et al., J. Neurosci. 25, 1219-1225, 2005), while mice lacking APP alone exhibit increased synapses and associated synaptic function (Priller et al., J. Neurosci. 26, 7212-7221, 2006). In contrast, overexpression of APP in transgenic mice results in deficits of synaptic transmission and learning (Saganich et al., J. Neurosci. 26, 13428-13436, 2006) and dendritic spine abnormalities (Spires et al., J. Neurosci. 25, 7278-7287, 2005).

Cleavage of APP results in the release of AICD, which has been shown to interact with Fe65 and Tip60 and has been suggested to function in nuclear signaling (Baeki et al., Cell 110, 55-67, 2002; Cao and Sudhof, 2001). Subsequent work by Cao and Sudhof (Cao et al., J. Biol. Chem. 279, 24601-24611, 2004) demonstrated that nuclear translocation of AICD may be dispensable, raising the possibility that AICD could function by modulating activation of Fe65 rather than functioning as a transcriptional regulator itself. Several studies have reported putative target genes differentially regulated by an AICD-containing complex, including the prostate cancer antimetastasis gene KAI1 (Baek et al., 2002), APP, GSK3b (Von Rotz et al., J. Cell Sci. 117, 4435-4448, 2004), neprilysin (Pardossi-Piquard et al., Neuron 46, 541-554, 2005), regulators of actin dynamics (Muller et al., Mol. Biol. Cell 18, 201-210, 2007), and the EGF receptor (Zhang et al., Proc. Natl. Acad. Sci. USA 104, 10613-10618, 2007). However, the exact role of AICD in transcriptional regulation of target genes remains controversial (Hebert et al., EMBO Rep. 7, 739-745, 2006; Chen and Selkoe, Neuron 53, 479-483, 2007; Pardossi-Piquard et al., 2007). Specifically, work by De Strooper and colleagues (Hebert et al., 2006) has shown that expression of several previously defined AICD target genes was at best indirectly and weakly affected by APP processing. Further, the role of AICD in regulating neprilysin expression (Pardossi-Piquard et al., 2005; Pardossi-Piquard et al., Neuron 53, 483-486, 2007) was not reproduced by another report (Chen and Selkoe, 2007). Finally, a recent study has shown that secreted APP ectodomain APPsa is sufficient to rescue several anatomical, behavioral, and electrophysiological abnormalities seen in APP-KO mice (Ring et al., J. Neurosci. 27, 7817-7826, 2007).

Although FAD genetics and mouse models have generated tremendous insights into AD pathogenesis, the vast majority of AD cases are sporadic with late-onset. A major genetic risk factor that was initially discovered in 1993 and has since been validated in numerous genetic studies is the presence of the ε4 allele of the apolipoprotein E (APOE) gene (Corder et al., Science 261, 921-923, 1993). ApoE is a major apolipoprotein in the brain and exists in three isoforms in humans (apoE2, apoE3, apoE4), each differing by a single amino acid (Mahley, Science 240, 622-630, 1988). In the brain, apoE/lipoprotein particles are produced primarily by astrocytes and are believed to deliver cholesterol and other lipids to neurons via lipoprotein receptors, namely members of the low-density lipoprotein receptor (LDLR) family (Herz and Bock, Annu. Rev. Biochem. 71, 405-434, 2002; Herz and Chen, Nat. Rev. Neurosci. 7, 850-859, 2006). Although the mechanisms underlying the pathogenic nature of apoE4 in sporadic AD are still poorly understood, several models have been proposed and supported by in vitro and in vivo studies. First, apoE interacts with Aβ, and apoE4 likely possesses greater ability to promote Aβ fibrillogenesis and amyloid plaque formation (Holtzman et al., Proc. Natl. Acad. Sci. USA 97, 2892-2897, 2000). Second, apoE facilitates Aβ clearance via apoE receptors expressed either in neurons (Zerbinatti and Bu, Rev. Neurosci. 16, 123-135, 2005) or in the blood-brain barrier (Zlokovic, Trends Neurosci. 28, 202-208, 2005). ApoE4 is less functional in Aβ clearance owing to its weaker affinity to Aβ (LaDu et al., J. Biol. Chem. 269, 23403-23406, 1994). Third, apoE4 may be toxic to neurons independently of Aβ aggregation and clearance (Huang, Curr. Opin. Drug Discov. Dev. 9, 627-641, 2006). It is possible that multiple pathways contribute to the pathogenic nature of apoE4 in AD.

Although the ε4 allele of the APOE gene was discovered as a strong genetic risk factor for late-onset AD over a decade ago (Corder et al., 1993), the mechanism by which apoE4 contributes to AD pathogenesis is still largely unclear. Furthermore, whether APP and apoE regulate common biological pathways is unknown. Cholesterol is an essential component of the cellular membrane and plays pivotal roles in development and maintenance of neuronal plasticity and function (Ledesma and Dotti, FEBS Lett. 580, 5525-5532, 2006). In the adult brain, neuronal cholesterol is supplied primarily by apoE/lipoprotein particles synthesized and secreted by glial cells (Pfrieger, Cell. Mol. Life Sci. 60, 1158-1171, 2003; Puglielli et al., Nat. Neurosci. 6, 345-351, 2003). Uptake of apoE/lipoprotein particles by neurons is mediated by lipoprotein receptors of the LDLR family (Herz and Bock, 2002). Endocytosed cholesterol-containing lipoprotein particles are hydrolyzed in neuronal lysosomes, allowing degradation of apoE and intracellular release of free cholesterol, which can be stored or incorporated into lipoprotein particles or cellular membranes. A function for LRP1 in brain apoE/cholesterol metabolism has been postulated (Pfrieger, 2003), but direct biological evidence has been lacking.

Cholesterol is an essential component of membranes and myelin sheathes and is crucial for synaptic integrity and neuronal functions (Pfrieger, 2003). Interestingly, an association between brain cholesterol metabolism and the risk of AD has been proposed (Shobab et al., Lancet Neurol. 4, 841-852, 2005). Early studies indicated that the use of statins, which inhibit cholesterol synthesis, are associated with a significant decrease in AD prevalence; however, several recent prospective studies do not support such a conclusion (Shobab et al., 2005). Additionally, the effect of cholesterol on the amyloidogenic processing of APP to Aβ remains controversial (Ledesma and Dotti, 2006). Intriguingly, apoE4 knock-in mice exhibit decreased brain cholesterol levels even though the peripheral cholesterol levels are increased (Hamanaka et al., Hum. Mol. Genet. 9, 353-361, 2000). A reduction of brain cholesterol levels is also observed in AD brains (Ledesma and Dotti, 2006). These disparate findings raise the need for an understanding of brain cholesterol metabolism and its potential dysregulation in AD.

The cell surface receptor LRP1, which is highly expressed in neurons in the brain, is critical for brain apoE/lipoprotein metabolism and synaptic functions (Bu, 2009. Nat Rev Neurosci 10:333-44; Liu et al. 2007. Neuron 56:66-78). LRP1 also plays important roles in neuronal survival (Hayashi et al, 2007. J Neurosci 27:1933-41) and Aβ clearance (Bu et al, 2006. Ann NY Acad Sci 1086:35-53). More importantly, LRP1 levels are reduced ˜50% in AD brains (Kang et al. 2000. J Clin Invest 106:1159-66). Restoring LRP1 expression in AD brains therefore represents a novel therapeutic strategy to combat AD. The present inventors have created an assay which allows the identification of pharmacological agents that can up-regulate LRP1 expression with minimal toxicity.

### SUMMARY

The inventors have discovered that the γ-secretase cleavage of APP regulates apoE and cholesterol metabolism in the central nervous system (CNS) via LRP1. The inventors have also established a biological linkage between APP and apoE, the two major genetic determinants of AD.

Specifically, the inventors have discovered that a cell surface receptor, LRP1, which is highly expressed in neurons in the brain, is essential for brain cholesterol and lipid metabolism. Deletion of the LRP1 gene in specific regions of mouse brain significantly impaired lipid metabolism and decreased brain cholesterol levels. These findings indicate that LRP1 expression in the brain can affect brain lipid and cholesterol levels. Because cholesterol and several other lipids are altered in AD as well as several other neurological disorders, modulation of LRP1 expression can be of therapeutic value to treatment of these neurological disorders.

The inventors further disclose an in vitro screen which can identify candidate compounds for the treatment of AD. The screen makes use of a labeled LRP1 ligand. In these screening methods, cells expressing LRP1 can be contacted with both a candidate compound and a labeled LRP1 ligand. The pharmaceutical activity of a candidate compound can be evaluated by determining the amount of labeled ligand associated with cells expressing LRP1 in the presence of the candidate compound. In some embodiments, the amount of labeled ligand associated with cells expressing LRP1 and treated with a candidate compound exceeds the amount of labeled ligand associated with cells expressing LRP1 but not treated with a candidate compound. In further embodiments, the cells expressing LRP1 can be hepatocytes, keratinocytes, embryonic cells, fibroblasts, macrophagess, Mueller glial cells, mammary epithelial cells, glioblastoma cells, retinal pigment epithelial cells, neurons, astrocytes, microglia, smooth muscle cells, endothelial cells or osteoblasts. In some embodiments, the cells expressing LRP1 can be human cells, mouse cells, rat cells, chicken cells, Xenopus cells, zebrafish cells, Drosophila cells and rabbit cells. In various embodiments, the cells can be U87 glioblastoma cells. In some embodiments, the LRP1 ligand can be any LRP1 such as, for example, an anti-LRP1 antibody, apoE, lipoprotein lipase, hepatic lipase, tPA, uPA, factor IXa, Factor VIIIa, factor VIIa, TFPI, matrix metalloproteinase-13 (MMP-13), MMP-9, spingolipid activator protein (SAP), pregnancy zone protein, alpha2-macroglobulin, complement C3, PAI-1, C1 inhibitor, anitthrombin III, heparin cofactor II, alpha1-antitrypsin, APP, thrombospondin-1, thrombospondin-2, Pseudomonas exotoxin A, rhinovirus, receptor-associated protein (RAP), lactoferrin, heat-shock protein 96 (HSP96), HSP90alpha and HIV-Tat protein. In some embodiments, the LRP1 ligand is RAP. In some embodiments, the LRP1 ligand can be an anti-LRP1 antibody. In further embodiments, the label can be any enzyme, radioisotope, fluorogen, fluorophore, chromogen, chromophore or other label known to skilled artisans. In some embodiments, the enzyme can be a peroxidase, a phosphatase, a galactosidase and a luciferase. In some embodiments, the radioisotope can be a ³²P, a ³³P, ³⁵S, a ¹⁴C, an ¹²⁵I, an ¹³¹I and a ³H. In some embodiments, the fluorophore can be a fluorescein, a rhodamine, an Alexa Fluor® (Invitrogen Corporation, Carlsbad Calif.), an IRDye® (LI-COR Biosciences, Lincoln, Nebr.), a coumarin, an indocyanine and a quantum dot. In other embodiments, the label can be a biotin, a digoxygenin, and a peptide comprising an epitope. In a some embodiment, the fluorophore is AlexaFluor488® (Invitrogen Corporation, Carlsbad Calif.). Measuring the amount of label associated with the cells can comprise any method thereof known to the ordinarily skilled artisan.

In one embodiment, linkage between APP processing and apoE/cholesterol metabolism is shown. Specifically, it is shown that a lack of either APP/APLP2 or PS1/PS2 leads to increased brain apoE/cholesterol catabolism.

In another embodiment, it is shown that the APP processing product, AICD, suppresses expression of the major apoE/lipoprotein receptor LRP1 by binding directly to its promoter following association with the adaptor protein Fe65. The defective apoE/cholesterol catabolism in APP/APLP2 and PS1/PS2 knockout cells was restored by forced expression of AICD. The inventors have discovered a biological function of APP in the regulation of brain apoE and cholesterol metabolism which offer new alternatives for the design of therapeutic strategies in the treatment of Alzheimer's Disease. This disclosure can also hold promise for the design of therapeutic strategies for the treatment of a variety of neurological diseases.

One embodiment is directed to the generation of Cre-lox conditional LRP1 forebrain knockout mice comprising crossing LRP1 floxP mice with αCamKII-Cre mice. Cre-lox conditional knockout mice are potentially useful for drug design in developing new compounds for the treatment of neurological diseases, including AD.

Another embodiment is directed to the enhanced expression and function of LRP1 by the deletion of APP and its homolog APLP2, or components of the γ-secretase complex.

One embodiment is directed to the suppression of the transcription of the LRP1 promoter by AICD together with Fe65 and Tip60.

One embodiment is directed to the regulation of apoE and cholesterol metabolism in the CNS by way of the modulation of LRP1.

Another embodiment is directed to the regulation of apoE and cholesterol metabolism in the CNS by way of the cleavage of APP by γ-secretase.

One embodiment of the present teachings discloses methods by which to down-regulate LRP1 expression and transport function. In a particular embodiment, the down-regulation can be achieved by the use of siRNA. In a further embodiment, LRP1 expression and transport function can be measured using a labeled LRP1 ligand. In some embodiments, the ligand can be a receptor associated protein, and the label can be a flourophore. In a particular embodiment, the receptor associated protein is RAP, and the flourophore is AlexaFluor488.

One embodiment of the present teachings discloses methods by which a compound that upregulates LRP1 expression and transport function is identified. In some embodiments, these methods comprise measuring LRP1 expression and transport function in cells after treatment with a pharmacological agent. In some embodiments, the cells can be U87 glioblastoma cells. In some embodiments, treatment with a pharmacological agent comprises incubating cells with the pharmological agent. In some embodiments, measuring LRP1 expression utilizes a fluorescently-labeled LRP1 ligand. In some embodiments, the fluorescently-labeled LRP1 ligand can be the LRP1 ligand RAP labeled with AlexaFluor488.

In one embodiment, the present teachings disclose high-throughput screening of compounds that upregulate LRP1 expression and transport function. In a specific embodiment, these methods comprise measuring LRP1 expression and transport function in cells after treatment with a pharmacological agent. In some embodiments, the cells can be U87 glioblastoma cells. In some embodiments, treatment with a pharmacological agent comprises incubating cells with the pharmological agent. In some embodiments, measuring LRP1 expression utilizes a fluorescently-labeled LRP1 ligand. In some embodiments, the fluorescently-labeled LRP1 ligand is RAP labeled with AlexaFluor488.

A further embodiment of the present teaching discloses in vivo screening of compounds that upregulate LRP1 expression and transport function. In some embodiments, it is determined whether a selected compound crosses the blood-brain barrier. In further embodiments, it is determined whether the selected compound is acceptably neuro-toxic. In a further embodiment, the bioavailability of the selected compound can be tested. The testing can comprise pharmacoknetic-pharmacodynamic modeling in mice. The pharmacoknetic-pharmacodynamic modeling can identify an optimal dosage and delivery method. In a further embodiment, the effects of the selected compound on LRP1 expression, Aβ accumulation, amyloid plaque formation and memory function is tested.

The present application also includes the following aspects:

Aspect 1. A method of identifying a compound for treating Alzheimer's disease, the method comprising:

a) forming an in vitro mixture comprising i) cells expressing LDL-receptor-related protein 1 (LRP1), ii) an LRP1 ligand comprising a label, and iii) a candidate compound; and

b) determining quantity of the label incorporated by the cells,

whereby the candidate compound is effective for treating Alzheimer's disease if the quantity of label incorporated by the cells exceeds that of a control in vitro mixture comprising cells expressing LRP1 and the LRP1 ligand, but not comprising the compound.

Aspect 2. A method according to aspect 1, wherein the cells expressing LRP1 are selected from the group consisting of hepatocyte cells, keratinocyte cells, embryonic cells, fibroblast cells, macrophage cells, Mueller glial cells, mammary epithelial cells, glioblastoma cells, retinal pigment epithelial cells, osteoblast cells, neurons, astrocytes, microglia, smooth muscle cells, endothelial cells.

Aspect 3. A method according to aspect 1, wherein the cells expressing LRP1 are selected from the group consisting of keratinocytes and glioblastoma cells.

Aspect 4. A method according to aspect 1, wherein the cells expressing LRP1 are selected from the group consisting of human cells, mouse cells, rat cells, chicken cells, Xenopus cells, zebrafish cells, Drosophila cells and rabbit cells.

Aspect 5. A method according to aspect 4, wherein the cells expressing LRP1 are selected from the group consisting of human cells and mouse cells.

Aspect 6. A method according to aspect 3, wherein the cells expressing LRP1 are U87 glioblastoma cells.

Aspect 7. A method according to aspect 1, wherein the LRP1 ligand is selected from the group consisting of apoE, lipoprotein lipase, hepatic lipase, tPA, uPA, factor IXa, Factor VIIIa, factor VIIa, TFPI, matrix metalloproteinase-13 (MMP-13), MMP-9, spingolipid activator protein (SAP), pregnancy zone protein, alpha2-macroglobulin, complement C3, PAI-1, C1 inhibitor, anitthrombin III, heparin cofactor II, alpha1-antitrypsin, APP, thrombospondin-1, thrombospondin-2, Pseudomonas exotoxin A, rhinovirus, receptor-associated protein (RAP), lactoferrin, heat-shock protein 96 (HSP96), HSP90alpha and HIV-Tat protein.

Aspect 8. A method according to aspect 7, wherein the LRP1 ligand is RAP.

Aspect 9. A method according to aspect 1, wherein the label is selected from the group consisting of an enzyme, a radioisotope, a fluorogen, fluorophore, a chromogen and a chromophore.

Aspect 10. A method according to aspect 9, wherein the enzyme is selected from the group consisting of a peroxidase, a phosphatase, a galactosidase and a luciferase.

Aspect 11. A method according to aspect 9, wherein the radioisotope is selected from the group consisting of a ³²P, a ³³P, ³⁵S, a ¹⁴C, an ¹²⁵I, an ¹³¹I and a ³H.

Aspect 12. A method according to aspect 9, wherein the fluorophore is selected from the group consisting of a fluorescein, a rhodamine, an Alexa Fluor®, an IRDye®, a coumarin, an indocyanine and a quantum dot.

Aspect 13. A method according to aspect 9, wherein the label is selected from the group consisting of a biotin, a digoxygenin, and a peptide comprising an epitope.

Aspect 14. A method according to aspect 12, wherein the fluorophore is AlexaFluor488.

Aspect 15. A method of treating a neurological disorder, the method comprising administering to a subject in need of treatment a therapeutically effective amount of an agent which increases expression of lipoprotein receptor LRP1.

Aspect 16. A method in accordance with aspect 15, wherein the neurological disorder is Alzheimer's disease.

Aspect 17. A method in accordance with aspect 15, wherein upon administration, the agent alters at least one neural or neuronal activity or property selected from the group consisting of: a) enhancing LRP1 activity; b) increasing brain cholesterol levels; and c) decreasing ApoE levels.

Aspect 18. A method in accordance with aspect 17, wherein administering the agent inhibits expression of at least one polypeptide selected from the group consisting of APP, APLP2, and a component of the γ-secretase complex.

Aspect 19. A method of inhibiting expression or function of LRP1 in a neuron, the method comprising contacting the neuron with an agent which forces expression of the APP intracellular domain (AICD).

Aspect 20. A method for inhibiting transcription in a cell of a nucleic acid sequence operably linked to an LRP1 promoter, the method comprising administering to a cell an effective amount of one or more of AICD, Fe65, and Tip60.

Aspect 21. A method for increasing cholesterol levels in the brain, the method comprising inhibiting expression of at least one gene selected from the group consisting of APP, APLP2, and a component of the γ-secretase complex.

Aspect 22. A method for the treatment of Alzheimer's disease, the method comprising inhibiting the expression of at least one gene selected from the group consisting of APP, APLP2, and a component of the γ-secretase complex.

Aspect 23. A method for the treatment of Alzheimer's disease, the method comprising administering to a subject in need of treatment an agent which increases LRP1 activity.

Aspect 24. A method for the treatment of Alzheimer's disease, the method comprising administering to a subject in need of treatment an agent which increases clearance of brain amyloid β-peptide.

Aspect 25. A method of screening a compound for treatment of Alzheimer's disease, the method comprising: a) administering a candidate compound to a laboratory mammal; and b) determining if LRP1 expression or activity increases in the brain of the mammal.

Aspect 26. A method of screening a compound for treatment of Alzheimer's disease in accordance with aspect 25, wherein determining if LRP1 expression increases comprises measuring brain LRP1 polypeptide levels with an antibody against LRP1.

Aspect 27. A method of screening a compound for treatment of Alzheimer's disease in accordance with aspect 25, wherein the mammal comprises a transgene, said transgene comprising an LRP1 promoter operably linked to a sequence encoding a reporter.

Aspect 28. A method of screening a compound for treatment of Alzheimer's disease in accordance with aspect 27, wherein the reporter is selected from the group consisting of a enzyme and a light-emitting polypeptide.

Aspect 29. A method of screening a compound for treatment of Alzheimer's disease in accordance with aspect 28, wherein the light-emitting polypeptide is selected from the group consisting of a luciferase and a fluorescent protein.

Aspect 20. A method of screening a compound for treatment of Alzheimer's disease in accordance with aspect 28, wherein the enzyme is selected from the group consisting of a horseradish peroxidase, an alkaline phosphatase, and a β-galactosidase.

Aspect 31. A method of screening a compound for treatment of Alzheimer's disease, the method comprising: a) administering a candidate compound to a laboratory mammal; and b) determining if expression or activity of an APP, a PS, or a combination thereof decreases in the brain of the mammal.

All publications and patent applications cited in this specification are herein incorporated by reference in their entireties, as if each individual publication or patent application were specifically and individually indicated to be incorporated by reference.

### BRIEF DESCRIPTION OF THE DRAWINGS

Those of skill in the art will understand that the figures, described below, are for illustrative purposes only. The figures are not intended to limit the scope of the present teachings in any way.

FIG. 1 shows the APP regulation of ApoE and cholesterol metabolism.

FIG. 2 shows densitometric analyses of western blots showing the regulation of LRP1 expression and function by amyloid precursor protein (APP) and its homolog (APLP2).

FIG. 3 shows densitometric quantification of western blots and double immunofluorescence staining showing LRP1 as a function of brain ApoE and cholesterol metabolism.

FIG. 4 demonstrates that the absence of LRP1 expression increases ApoE half-life.

FIG. 5 depicts densitometric analyses of western blots which show the regulation of LRP1 expression by gamma-secretase.

FIG. 6 depicts densitometric analyses of western blots which show the regulation of LRP1 expression by AICD in the absence of APP/APLP2 or PS1/2.

FIG. 7 depicts densitometric analyses of western blots which show that AICD binds to and suppresses LRP1 promoter activation.

FIG. 8 shows that AICD rescues ApoE and cholesterol defects in cells deficient in PS or APP.

FIG. 9 shows micrographs which demonstrate that spine degeneration in LRP-FB-KO mice brains. Representative pyramidal neurons from layer 5 of the cortex are shown.

FIG. 10 shows a densitometric analysis of a western blot and a scatter plot, all of which show impaired synaptic integrity and functions in LRP-FB-KO mice.

FIG. 11 contains a schematic diagram, western blots, and double immunofluorescence staining micrographs, all of which show that LRP knockdown in primary neurons leads to caspase-mediated apoptosis.

FIG. 12 shows fluorescence images and quantitative data from cells in which LRP1 knockdown was achieved via siRNA.

FIG. 13 shows fluorescence images and quantitative data from cells in which LRP1 is overexpressed.

FIG. 14 demonstrates that knockout of BACE1 does not affect LRP1 expression and/or function.

FIG. 15 shows that both LRP1 expression and function are increased in PS1-KO MEF cells.

FIG. 16 demonstrates that LRP1 expression and function are increased in Nicastrin-KO MEF cells.

### DETAILED DESCRIPTION

The present disclosure teaches the upregulation of LRP1 expression and function as a method for treatment of Alzheimer's disease and other neurological disorders. The disclosure also teaches means by which compounds may be screened to identify candidates for the treatment of AD.

One embodiment is directed to a mechanism by which the C-terminal fragment of APP, named AICD, modulates brain apoE and cholesterol metabolism by directly regulating the expression and function of the lipoprotein receptor LRP1. Knockout of APP/APLP2 or components of the gamma-secretase complex significantly affected the expression of LRP1 as well as apoE and cholesterol levels, and these alterations were partially restored by forced expression of AICD. The inventors have also discovered that AICD, together with the adaptor proteins Fe65 and Tip60, regulates LRP1 promoter function. The inventors have established a strong biological relationship between APP processing and apoE/cholesterol metabolism with significant relevance for the pathogenesis of AD.

The present inventors have shown that deletion of LRP1 in forebrain neurons of adult mice significantly increases brain apoE and decreases brain cholesterol levels. Previous studies have shown that overexpression of an LRP1 minireceptor in the brain results in a decrease in brain apoE level (Zerbinatti et al., J. Biol. Chem. 281, 36180-36186, 2006). The inventors have thus discovered a role for LRP1 in brain apoE and cholesterol metabolism and establish LRP1 as a neuronal receptor essential for the proper endocytosis and catabolism of apoE.

In the present application, results are shown which support a role for γ-secretase-dependent APP processing in the regulation of brain cholesterol levels via transcriptional repression of LRP1. The present findings do not exclude the possibility that APP itself or other APP processing products (e.g., soluble APP and AR) may also regulate LRP1 expression and function, however, these findings show that LRP1 expression and apoE/cholesterol metabolism are unchanged in BACE1 knockout mice argues against a role for Aβ in regulating LRP1 expression. Supporting an AR-independent function of γ-secretase, recent work has demonstrated that a complete loss of presenilin function in the forebrain leads to memory deficits, synaptic dysfunction, and neurodegeneration without generation of amyloid plaques (Saura et al., Neuron 42, 23-36, 2004). These results demonstrate that at least some of the neurodegenerative pathology seen in AD might result from partial loss of γ-secretase activity or AICD nuclear signaling functions independent of Aβ production (Shen and Kelleher, Proc. Natl. Acad. Sci. USA 104, 403-409, 2007). A recent study demonstrates a role for Aβ in regulating cholesterol biosynthesis and sphingomyelin degradation (Grimm et al., Nat. Cell Biol. 7, 1118-1123, 2005). Because AICD did not completely restore apoE and cholesterol levels in PS-DKO cells, it is possible that other γ-secretase cleavage products may also regulate apoE/cholesterol metabolism via LRP1-dependent and/or independent mechanisms. Regulation of cholesterol synthesis by AR, as well as AICD-mediated modulation of LRP1 expression, are likely to be key events in the proper maintenance of brain cholesterol levels.

The inventors of the present application have discovered the role of APP in modulating brain apoE and cholesterol homeostasis. An important role of the APP processing product AICD in regulating the promoter activity of LRP1, an essential lipoprotein receptor for brain apoE and cholesterol metabolism, has also been discovered. These findings regarding APP biological function and its potential implications for neuronal dysfunction in AD can lead to the design of better therapeutic strategies to treat AD and other neurological disorders.

Using a conditional knockout mouse model, the inventors provide in vivo evidence that LRP1 is a major cholesterol receptor in the brain. The inventors have also found that altering the expression levels of LRP1, APP, or PS all affect the brain levels of apoE and cholesterol.

The inventors have discovered that altering the expression and function of LRP1, APP, and PS can directly impact brain lipoprotein/cholesterol metabolism. The inventors of the present application have discovered that altering the expression of LRP1, APP, and PS can be explored as a way to increase or decrease brain apoE and cholesterol in neurological disorders. For example, in one embodiment, cholesterol level is decreased in AD and therefore an increase in LRP1 expression can be a way to restore cholesterol metabolism.

In another embodiment, the present inventors disclose the removal of amyloid beta peptide, a sticky peptide deposited in AD brain and a cause of AD pathology, by increasing LRP1 expression.

For the present application, studies were carried out using the materials and experimental procedures described below.

The methods and compositions described herein utilize laboratory techniques well known to skilled artisans and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999. Pharmaceutical methods and compositions described herein, including methods for determination of therapeutically effective amounts, and terminology used to describe such methods and compositions, are well known to skilled artisans and can be adapted from standard references such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003.

Materials

Human recombinant RAP was expressed in a glutathione S-transferase expression vector and isolated as described previously (Bu et al., J. Biol. Chem. 268, 13002-13009, 1993). All tissue culture media and serum were from Sigma. Anti-APP C-terminal antibody was purchased from Invitrogen; anti-Fe65 was from Abcam; anti-Tip60 was from Calbiochem; anti-actin was from Sigma; and anti-NeuN was from Chemicon. In-house anti-LRP1 and anti-LDLR antibodies have been described previously (Bu et al., EMBO J. 14, 2269-2280, 1995; Li et al., J. Cell Sci. 118, 5305-5314, 2005; Zerbinatti et al., Proc. Natl. Acad. Sci. USA 101, 1075-1080, 2004). Peroxidase-labeled anti-mouse antibody and ECL system were from GE Healthcare. Carrier-free Na¹²⁵I was purchased from Perkin Elmer Lifescience. The γ-secretase inhibitors L685,458 and DAPT were from Calbiochem, and DFK167 was from Enzyme Systems.

Animals and Tissue Preparation

LRP1 forebrain knockout mice were generated by breeding the LRP1 loxP mice (Rohlmann et al., J. Clin. Invest. 101, 689-695, 1998) with χ-calcium-calmodulin-dependent kinase II-driven Cre recombinase mice (Tsien et al., Cell 87, 1317-1326, 1996). Littennates of LRP1 forebrain knockout (LRP1^{flox+/+,} Cre^{+/-}) or WT controls (LRP1^{flox+/+}, Cre^{-/-}) at 11 months of age were used for western blotting, immunofluorescence staining, and apoE/cholesterol assays. APP-KO, APP/APLP2-DKO, and WT littermate control mice have been described previously and were used within 24 hr after birth due to potential lethality of the APP/APLP2-DKO mice. APP-KO mice were also analyzed at 4 months of age. PS1/2-DKO mice were generated by Cre-lox conditional deletion of the PS1 gene in forebrain of the PS2-KO mice (Feng et al., Proc. Natl. Acad. Sci. USA 101, 8162-8167, 2004) and were used at 4 months of age. BACE1-KO mice (Luo et al., Nat. Neurosci. 4, 231-232, 2001) were described in previous work and were used at 2 months of age. Animals were perfused with PBS-heparin (3 units/ml), and brain tissues were dissected and kept frozen at −80° C. until further analysis. All animal procedures were approved by the Animal Study Committee at Washington University School of Medicine and in accordance with the regulations of the American Association for the Accreditation of Laboratory Animal Care.

Reverse Transcriptase Real-Time PCR

Total RNA was isolated from tissues or cells using the SV Total RNA Isolation System (Promega) and subjected to DNase I digestion to remove contaminating genomic DNA. Total RNA was dissolved in nuclease-free water and stored at −80° C. Reverse transcription was performed using a SuperScript II RNase H-reverse transcriptase (Invitrogen), and the reaction mix was subjected to quantitative real-time PCR to detect levels of the corresponding actin, LRP1, or LDLR. The set of actin primers was used as an internal control for each specific gene amplification. The relative levels of expression were quantified and analyzed by using Bio-Rad iCycler iQ software. The real-time value for each sample was averaged and compared using the C_{T} method, where the amount of target RNA (2-^{eeCT}) was normalized to the endogenous actin reference (ΔC_{T}) and related to the amount of target gene in tissue cells, which was set as the calibrator at 1.0.

ApoE ELISA

The sandwich ELISA for mouse apoE has been described previously (Wahrle et al., J. Biol. Chem. 279, 40987-40993, 2004). Briefly, 96-well plates were coated overnight with apoE antibody (WU E4), washed with PBS, blocked with 1% milk in PBS, and then washed again. Cells or brain samples were sonicated in 5 M guanidine HCI with 1× Complete protease inhibitor mixture (Roche Applied Science), debris was pelleted by centrifugation at 10,000×g, and the supernatant was diluted in 0.1% BSA, 0.025% Tween 20 in PBS. Following sample incubation, the plate was washed, and 3 μg/well of biotinylated goat anti-apoE (Calbiochem) was added. After incubation with the secondary antibody, the plate was washed, and poly-horseradish peroxidase streptavidin (Pierce) was added at 1:6000 dilution and incubated. The plate was then washed, developed with tetramethylbenzidine (Sigma), and read at 650 nm with a Biotek 600 plate reader (Bio-Tek Instruments).

Cholesterol Analyses

Cells or brain samples were prepared for cholesterol analysis by sonication in PBS with 1× Complete protease inhibitor mixture. The homogenized whole-cell or brain suspension was then subjected to enzymatic analysis for total cholesterol using the Amplex Red Cholesterol Kit (Invitrogen) (Wahrle et al., 2004).

Immunofluorescence Staining

Frozen tissue sections were blocked with 0.1% Tween 20, 5% BSA in PBS for 30 min and stained for 2 hr at room temperature with anti-LRP1 antibody. Primary antibody was then visualized using Alexa 488-labeled goat anti-mouse secondary antibody (Invitrogen). Neurons were counterstained with anti-NeuN (Calbiochem) and Alexa 633-labeled secondary antibody (Invitrogen). Fluorescent images were captured with a confocal microscope (Olympus Fluoview 500).

Chromatin Immunoprecipitation

ChIP assays were performed using a chromatin immunoprecipitation (ChIP) assay kit (Upstate) according to the manufacturer's instructions with minor modifications. Briefly, brain tissue from WT C57BL/6J mice were minced into small pieces with a razor and 1% formaldehyde was added directly to the tissue mixture to crosslink proteins to DNA. Tissue was then lysed in SDS lysis buffer and sonicated to shear DNA to a size range of 200-1000 bp. After centrifugation, the supernatant was diluted 10-fold in ChIP dilution buffer and incubated overnight at 4° C. with anti-APP, anti-Fe65, anti-Tip60, or normal rabbit IgG. Protein A-agarose beads were used to immunoprecipitate the antibody/protein/DNA complexes. After washing, the complex was incubated at 65° C. for 4 hr to reverse the protein/DNA crosslinks. The DNA was then purified using PCR Purification kit (QIAGEN) and used as template for PCR amplification. LRP1-F (5′-TCGGGTGTCCCTGTTTAC-3′; SEQ ID NO: 1) and LRP1-R (5′-GAAAGCGGTCCAAGAGTG-3′; SEQ ID NO: 2) primers were used to amplify the LRP1 promoter by RT-PCR. LRP1-F (5′-GGGAGCCTGAAA TCCTAGAG-3′; SEQ ID NO: 3) and LRP1-R (5′-GGAAAGCGGTCCAAGAGTG-3′; SEQ ID NO: 4) primers were used to amplify LRP1 promoter by real-time PCR. Primers for HES1 promoter amplification are as follows: HES1-F, 5′-CGTGTCTCTTCCTCCCATTG-3′; SEQ ID NO: 5; HES1-R, 5′-GATCCAGTGTGATCCGCAGG-3′; SEQ ID NO: 6. PCR products were resolved on 2% agarose gels and visualized by ethidium bromide staining.

Luciferase Assay

BHK570 cells or MEF APP/APLP2-DKO cells were transfected with the appropriate cDNAs: empty vector (pGL3-luc), LRP1 promoter-luc, AICD, and/or Fe65. A β-gal reporter cDNA was co-transfected to normalize data for transfection efficiency. Twenty-four hours after transfection, cells were rinsed, gently scraped into PBS (pH 7.4), and pelleted. Cells were then lysed in lysis buffer, and the luciferase activity and β-gal activity were measured by the Luciferase Assay System and β-gal Assay System following the manufacturer's instructions (Promega).

Statistical Analysis

All quantified data represent an average of at least triplicate samples. Error bars represent standard error of the mean. Statistical significance was determined by Student's t test, and p<0.05 was considered significant.

The detailed description set forth above is provided to aid those skilled in the art in practicing the present teachings. However, the teachings described and claimed herein are not to be limited in scope by the specific embodiments herein disclosed. Any equivalent embodiments are intended to be within the scope of this invention. Various modifications of the teachings which do not depart from the spirit or scope of the present inventive discoveries, in addition to those shown and described here in, will become apparent to those skilled in the art from the foregoing description. Such modifications are also intended to fall within the scope of the appended claims.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### APP and APLP2 Regulate Brain ApoE and Cholesterol Metabolism

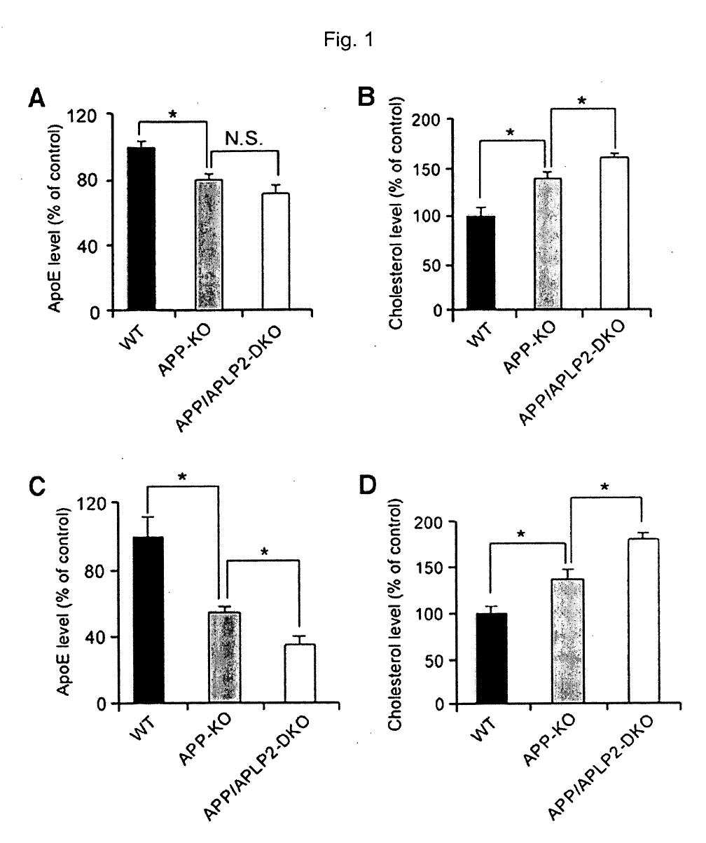

This example illustrates regulation of ApoE and cholesterol metabolism by APP. To examine whether APP processing and apoE/cholesterol metabolism are functionally related, cellular apoE and cholesterol levels in WT, APP knockout (APP-KO), or APP/APLP2 double-knockout (APP/APLP2-DKO) mouse embryonic fibroblasts (MEFs) was examined. To minimize potential clonal effects when MEF cells were established, APP-KO MEF cells stably retransfected with APP695 cDNA were used as WT controls. The APP-KO and APP/APLP2-DKO cells displayed a significant decrease in apoE levels and a concomitant increase in cholesterol levels compared to WT controls (see e.g., FIGS. 1A and 1B). The apoE and cholesterol levels that were measured likely reflect a combination of those derived from both serum and cells. These results show that lack of APP or APP/APLP2 leads to increased catabolism of apoE/lipoprotein, resulting in increased intracellular cholesterol levels (Mahley, 1988).

To assess whether these changes also occur in vivo, brain apoE and cholesterol levels in WT, APP-KO, and APP/APLP2-DKO mouse brain were compared. ApoE levels were decreased by about 50% in APP-KO and further decreased in APP/APLP2-DKO when compared to WT littermate controls (see e.g., FIG. 1C). A corresponding increase in cholesterol levels was also observed in APP-KO and APP/APLP2-DKO mouse brain when compared to WT controls (see e.g., FIG. 1D).

### Example 2

### APP and APLP2 Regulate LRP1 Expression and Function

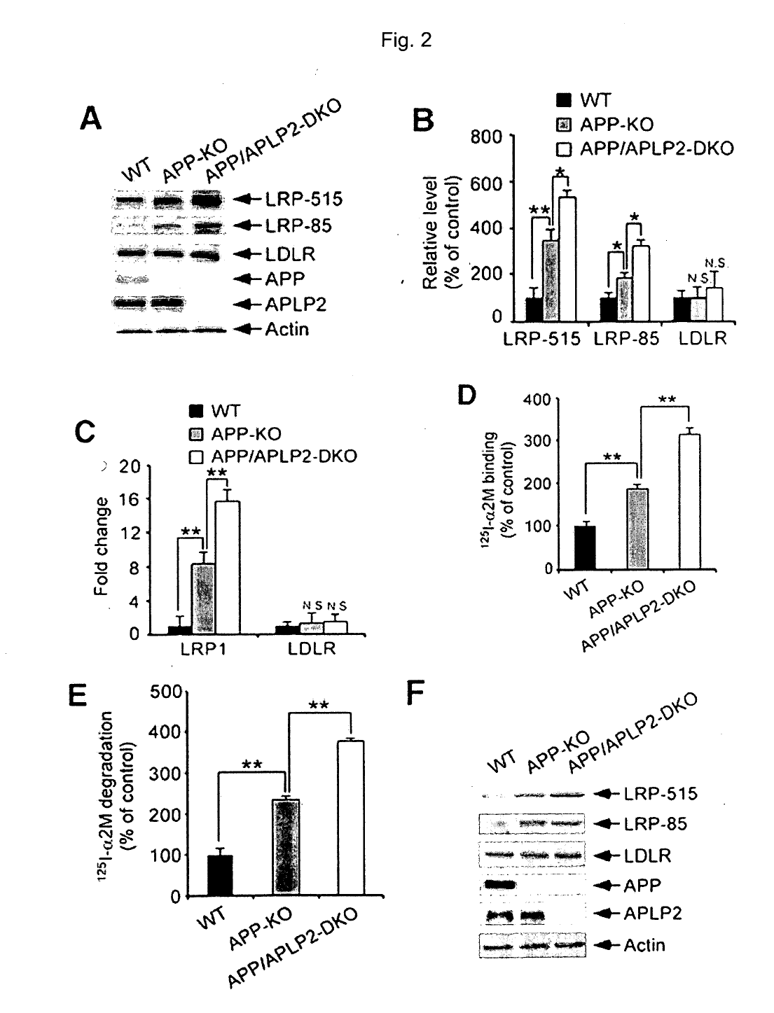

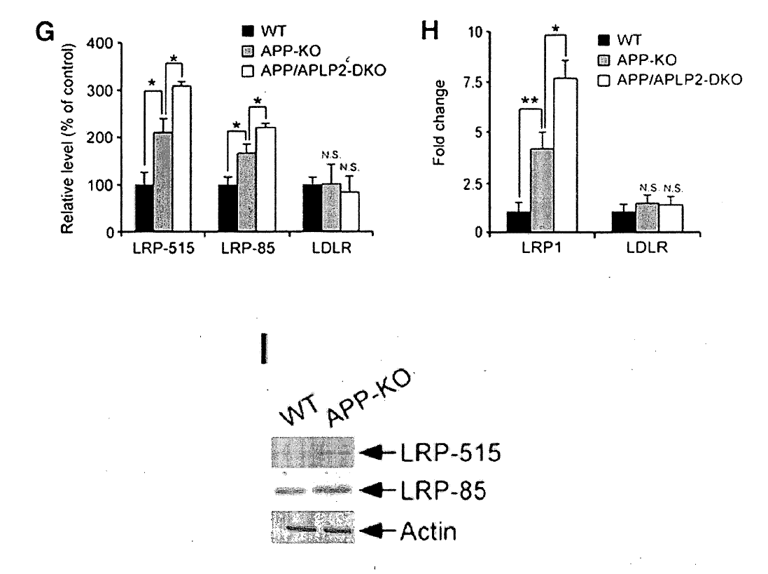

Because of the increased catabolism of apoE/lipoprotein by APP-KO and APP/APLP2-DKO cells, experiments were carried out to examine whether apoE receptor levels might be up-regulated in these cells. To test this possibility, expression levels of two major brain apoE receptors, LRP1 and LDLR (Herz and Bock, 2002), in WT, APP-KO, and APP/APLP2-DKO MEF cells were compared. Western blotting using two different antibodies to LRP1 showed that LRP1 levels were significantly increased in APP-KO MEF cells and in APP/APLP2-DKO MEF cells compared to WT MEF cells (see e.g., FIGS. 2A and 2B). The expression of LDLR was not altered by APP/APLP2 deletion.

To analyze whether changes in LRP1 expression were at the transcriptional or posttranscriptional level, LRP1 mRNA levels were compared by real-time PCR. LRP1 mRNA levels were significantly increased in both APP-KO and APP/APLP2-DKO MEF cells when compared to WT control cells (see e.g., FIG. 2C). To investigate whether changes in LRP1 expression also correlate with changes in LRP1 function, binding and endocytosis of α2-macroglobulin (α2M), a high-affinity ligand for LRP1 (Herz and Bock, 2002) was analyzed. Ligand-binding assays using ¹²⁵I-α2M demonstrated increased binding capacity in APP-KO and APP/APLP2-DKO MEF cells when compared to WT control cells (see e.g., FIG. 2D). Similar increase was seen when ¹²⁵I-α2M uptake and degradation were analyzed (see e.g., FIG. 2E).

To examine whether the in vitro findings were reproduced in vivo, LRP1 expression in the brains of newborn APP-KO, APP/APLP2-DKO and littermate control mice were compared. LRP1 expression, but not LDLR expression, was significantly increased in APP-KO and APP/APLP2-DKO mouse brains when compared to WT controls both at the protein (see e.g., FIGS. 2F and 2G) and mRNA levels (see e.g., FIG. 2H). A similar increase in LRP1 expression was also observed in the brain of APP-KO mice at 4 months of age. These results demonstrate that LRP1 expression and function are modulated by APP/APLP2 or their processing products and show that APP/APLP2 regulate brain apoE and cholesterol metabolism via regulation of LRP1 levels.

### Example 3

### LRP1 Modulates Brain ApoE/Lipoprotein Metabolism

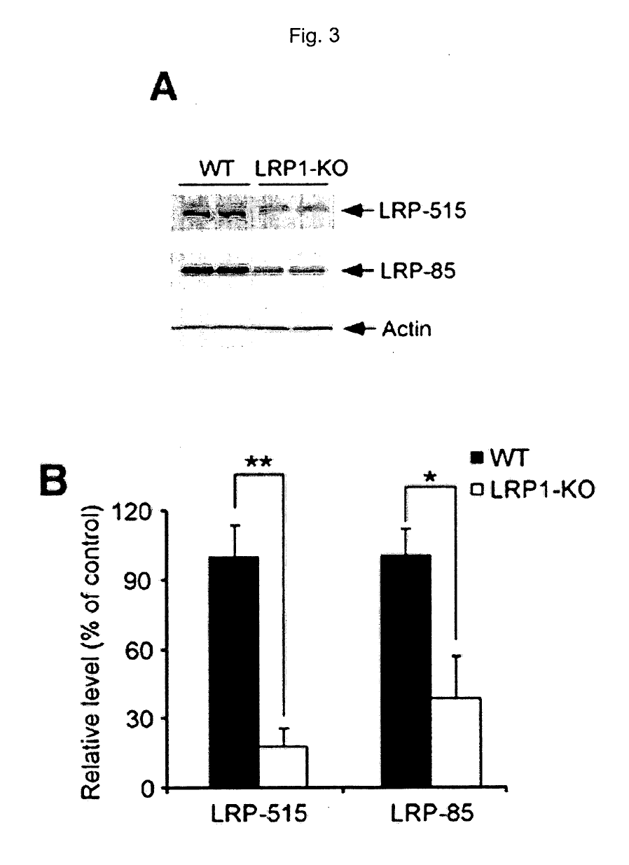

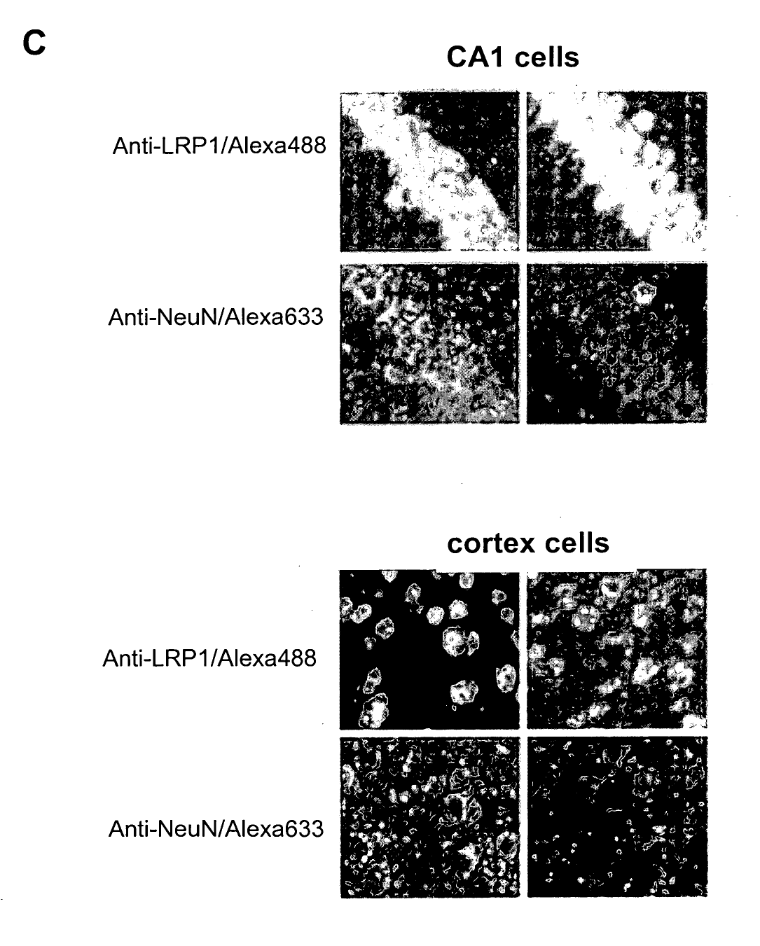

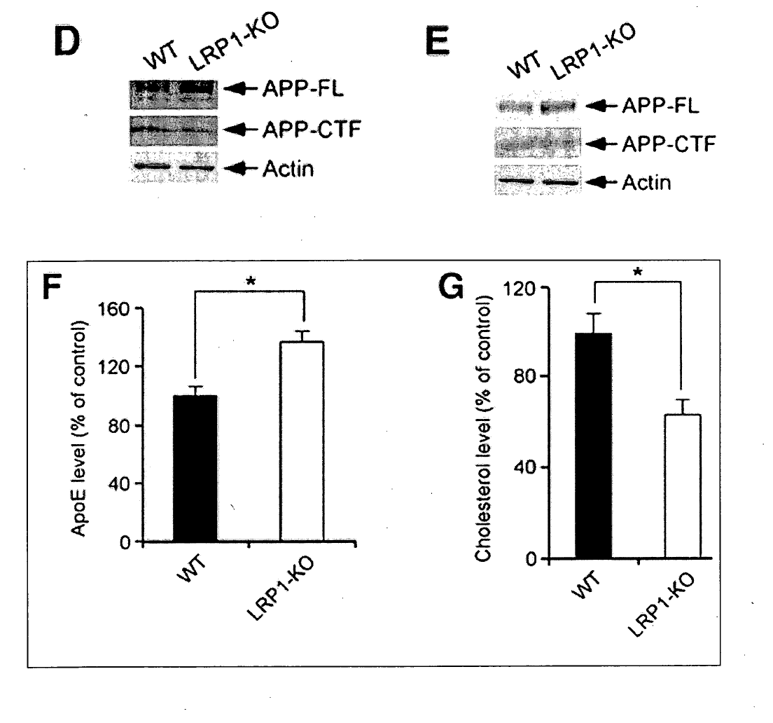

In an effort to provide in vivo evidence as to whether LRP1 regulates brain apoE and cholesterol metabolism, conditional LRP1 forebrain knockout mice were generated by crossing LRP1 floxP mice (Rohlmann et al., 1998) with αCamKII-Cre mice (Tsien et al., 1996). LRP1 expression was significantly decreased in the forebrain as determined by immunoblotting using antibodies directed against either the 515 kDa subunit or the 85 kDa subunit (see e.g., FIGS. 3A and 3B). Remaining LRP1 expression detected by Western blotting likely represents expression in glial cells (Moestrup et al., Cell Tissue Res. 269, 375-382, 1992). By double immunofluorescence staining using LRP 1-specific antibody and NeuN antibody, it was confirmed that LRP1 expression was nearly abolished in CA1 neurons of the hippocampus and in pyramidal neurons of the frontal cortex (see e.g., FIG. 3C).

To examine how LRP1 deletion affects APP levels and processing the steady-state levels of the full-length APP and APP C-terminal fragment (CTF) were compared. It was found that deletion of LRP1 slightly increased the steady-state levels of full-length APP while it decreased the levels of APP-CTF both in LRP1-KO MEF cells (see e.g., FIG. 3D) and in mouse brain (see e.g., FIG. 3E). This result shows that stabilization of APP at the cell surface is due to reduced endocytosis (Cam et al., J. Biol. Chem. 280, 15464-15470, 2005).

To evaluate the impact of LRP1 deletion on apoE and cholesterol metabolism, apoE and cholesterol levels in the forebrain of LRP1-KO and WT littermate controls were compared. It was found that while apoE levels were significantly increased (see e.g., FIG. 3F), cholesterol levels were decreased (see e.g., FIG. 3G), in the LRP1-KO mice. This result shows impaired catabolism of apoE/lipoprotein particles.

### Example 4

### LRP1 Modulates Brain ApoE/Lipoprotein Metabolism

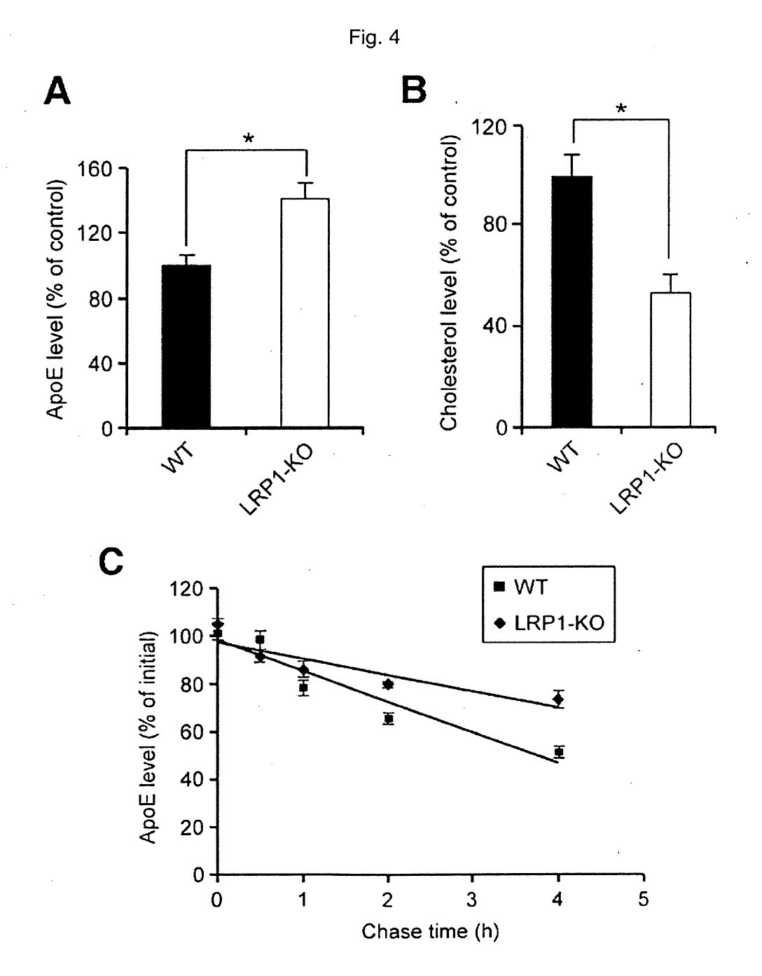

This example illustrates the changes in apoE and cholesterol levels in the absence of LRP1 in LRP1-KO MEF cells (see e.g., FIGS. 4A and 4B). The half-life of apoE is significantly increased in LRP1-KO MEF cells, demonstrating that in the absence of LRP 1, apoE catabolism is decreased (see e.g., FIG. 4C). ApoE mRNA levels were not changed between WT and LRP1-KO MEF cells (see e.g., FIG. 4D) or brain tissues (see e.g., FIG. 4E). These results demonstrate that LRP1 is a bona fide apoE/lipoprotein receptor that regulates apoE and cholesterol metabolism in the brain.

### Example 5

### Aβ is not Required for LRP1 Regulation

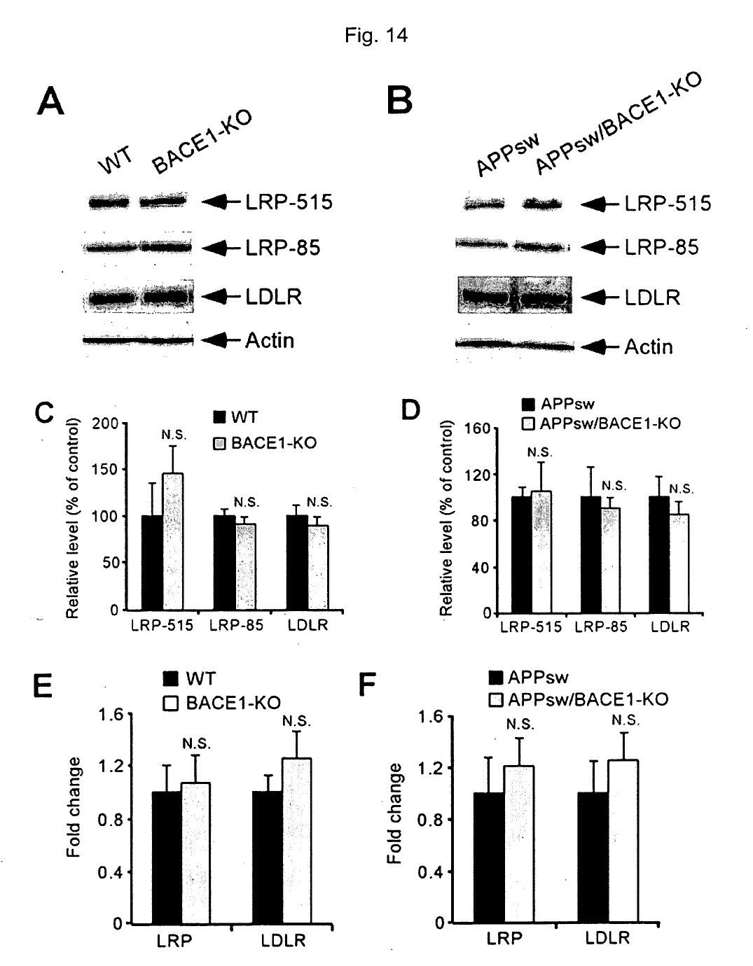

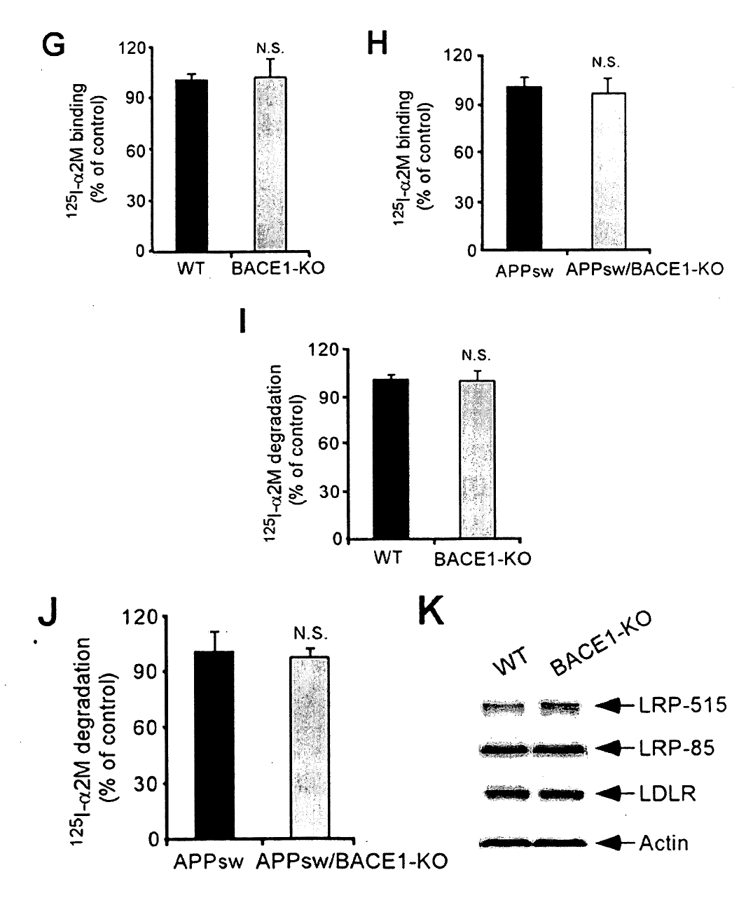

This example illustrates the results of a study that was designed to evaluate the possibility that Aβ may be the processing product of APP that regulates LRP1 expression levels. Because β-secretase BACE1 is necessary for Aβ production (Luo et al., 2001), the effects of BACE1 knockout on LRP1 expression was evaluated. This example shows that deficiency of BACE1 in either MEF cells or in mouse brain does not alter LRP1 expression or function (see e.g., FIG. 14). This example shows that there is no difference in LRP1 expression in MEF cells overexpressing a human APP bearing the Aβ-overproducing Swedish mutation (APPsw) in either the WT background (high Aβ levels) or BACE1-KO background (no Aβ) (see e.g., FIG. 14). These results show that Aβ production is not required for LRP1 regulation.

### Example 6

### γ-Secretase Activity Regulates LRP1 Expression and Function

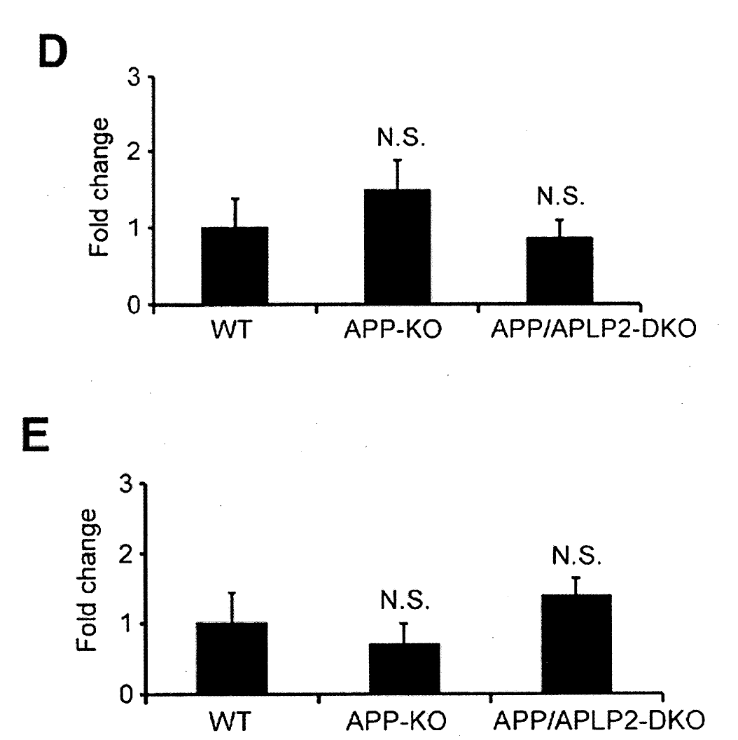

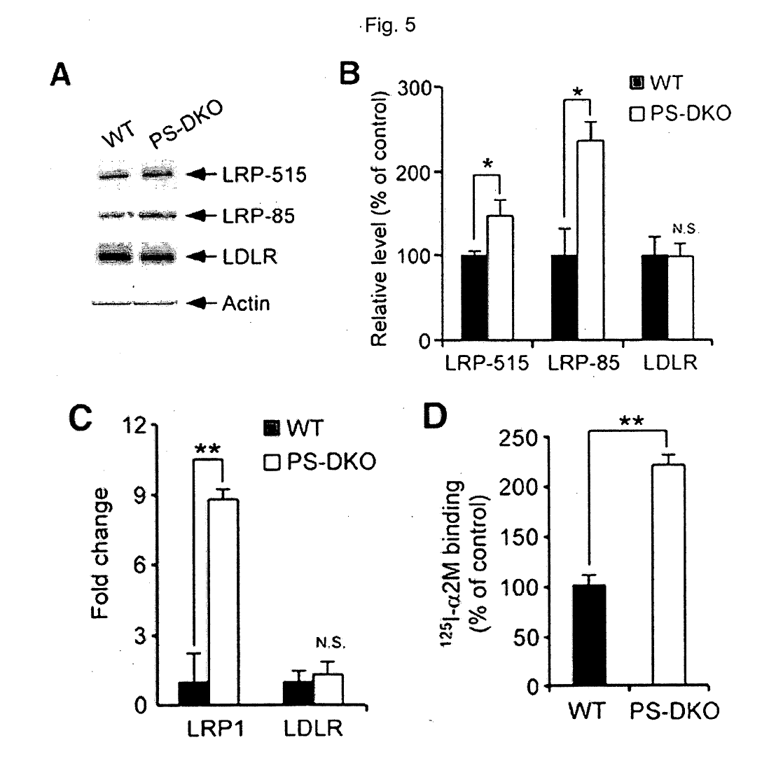

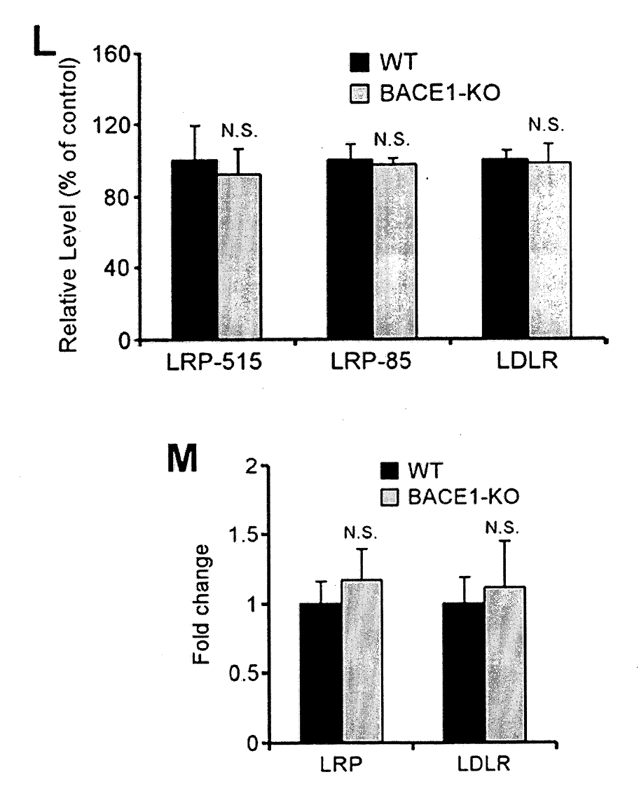

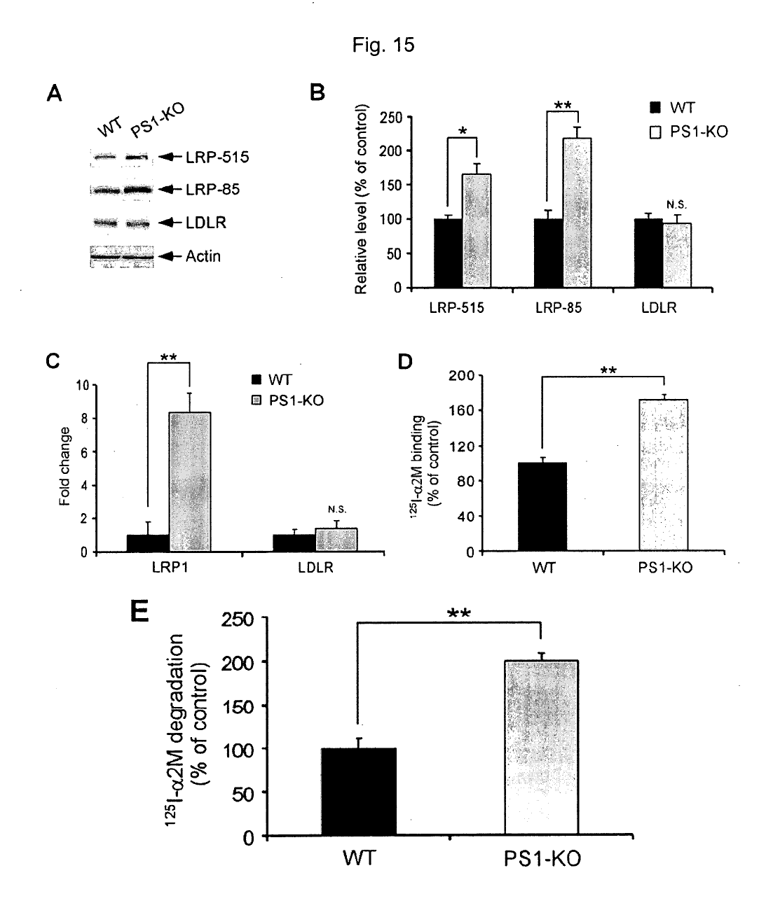

This example illustrates the results of experiments designed to examine whether γ-secretase cleavage is required for APP-mediated regulation of LRP1 expression. LRP1 expression in WT MEF cells was compared to those lacking presenilin (PS), an essential component of the γ-secretase complex. Western blotting showed that LRP1 expression was significantly increased in PS1/2 double-knockout (PS-DKO) MEF cells (see e.g., FIGS. 5A and 5B) when compared to WT MEF cells, while the expression of the LDLR was not affected. A similar increase in LRP1 expression was seen in PS1-KO MEF cells (see e.g., FIG. 15). Real-time PCR analysis confirmed an increase in LRP1 expression at the mRNA levels (see e.g., FIGS. 5C and 15). PS-DKO MEF cells also showed increased binding and degradation of the LRP1 ligand ¹²⁵I-α2M when compared to WT MEF cells (see e.g., FIGS. 5D, 5E, and 15).

To test whether LRP1 expression was altered in the absence of the γ-secretase function in vivo, brain tissues from PS-DKO, PS1-KO, and their littermate controls were analyzed for LRP1 expression by western blotting and real-time PCR. The loss of γ-secretase function in PS-DKO mouse brains was demonstrated by an accumulation of APP-CTF, a substrate for γ-secretase (see e.g., FIG. 5F). These results show that expression of LRP1 was significantly increased in PS-DKO (see e.g., FIGS. 5F-5H) and PS1-KO (see e.g., FIG. 15) mouse brains.

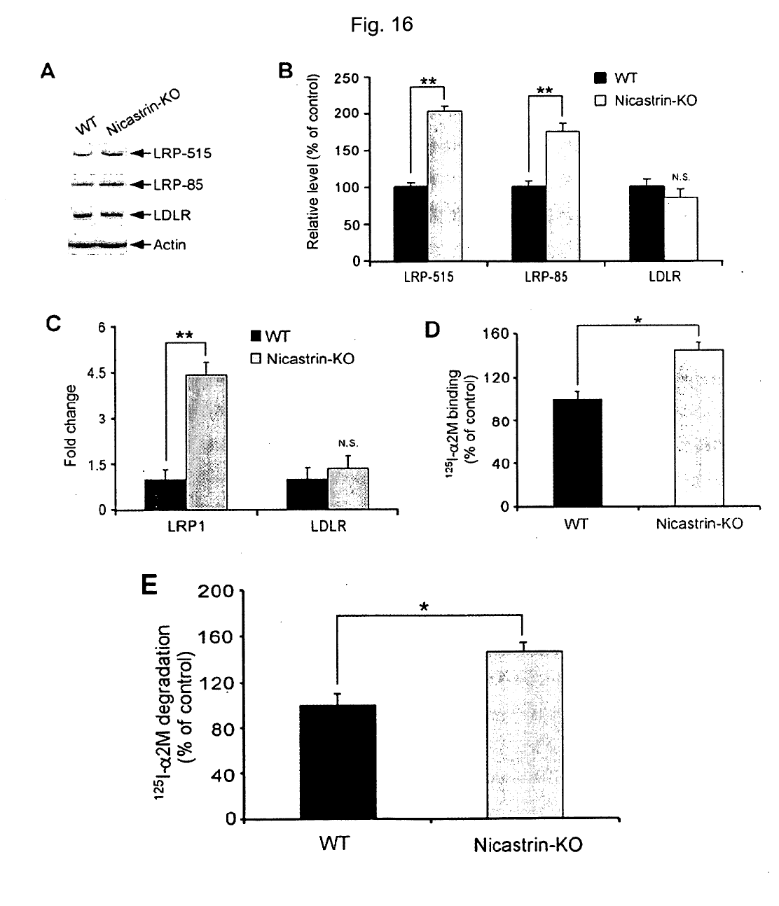

The requirement of γ-secretase activity in LRP1 regulation was further verified by two alternative approaches. MEF cells lacking nicastrin, another essential component of the γ-secretase complex (Selkoe and Kopan, 2003), also showed increased LRP1 expression and function compared to WT control MEF cells (see e.g., FIG. 16). Furthermore, treatment with three distinct γ-secretase inhibitors (L685,458, DAPT, DFK) enhanced LRP1 expression in WT MEF cells (see e.g., FIGS. 5I and 5J). These results demonstrate that γ-secretase activity regulates LRP1 expression and function both in vitro and in vivo.

### Example 7

### AICD Rescues LRP1 Expression in the Absence of APP/APLP2 or PS Expression

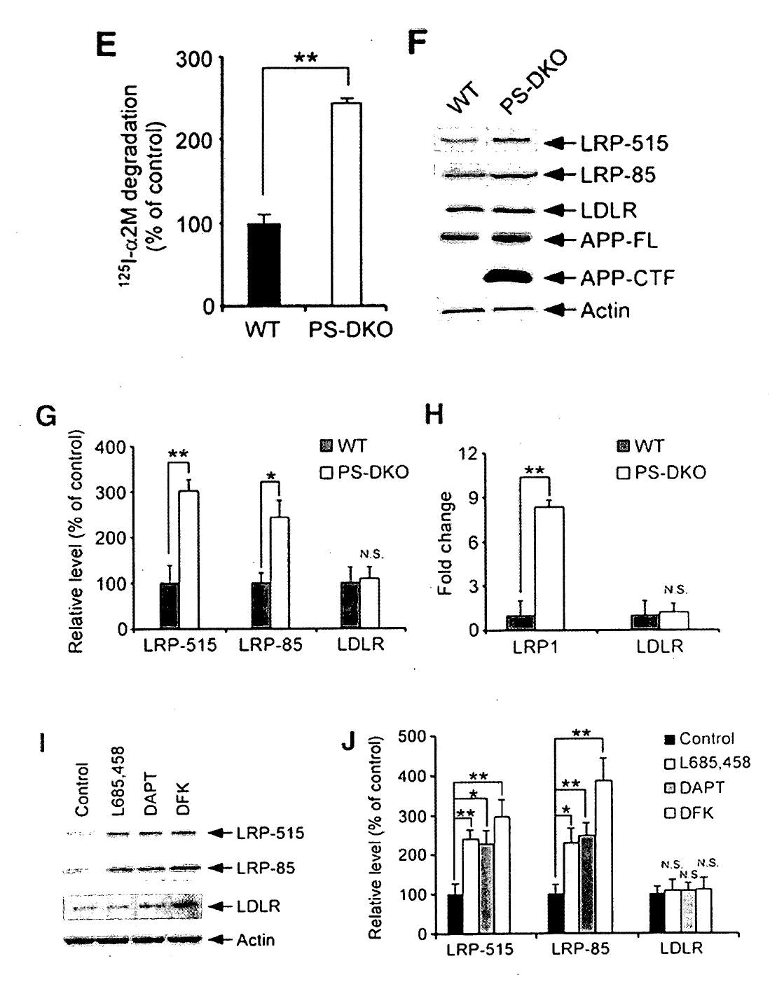

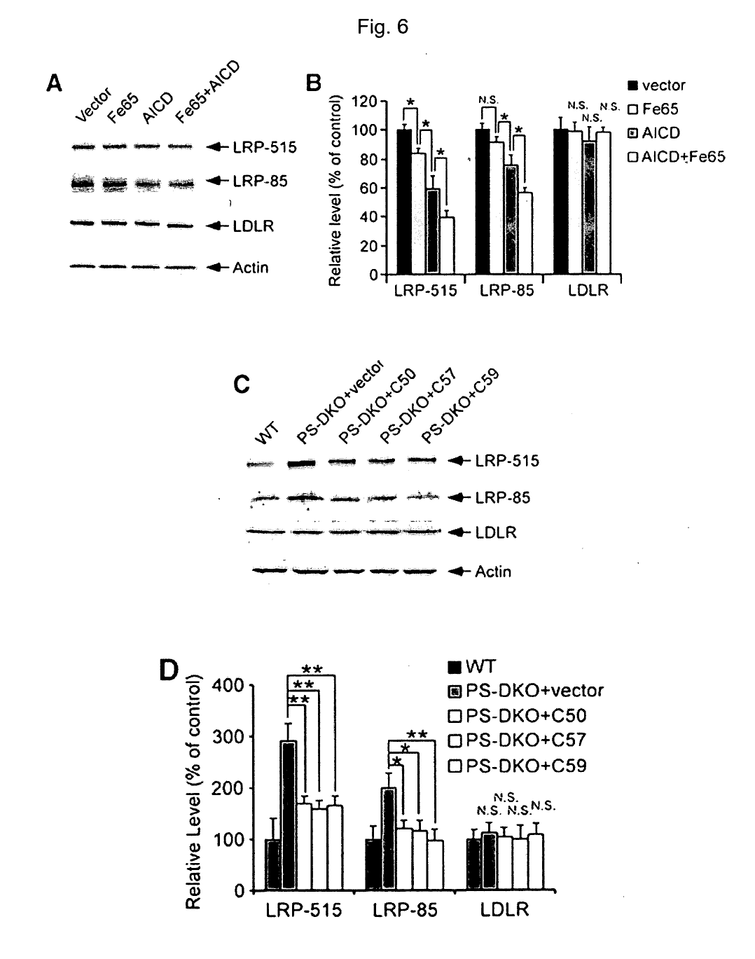

To directly address whether the γ-secretase product, AICD, is involved in LRP1 regulation, the effects of AICD forced expression on LRP1 expression were analyzed. Transient transfection of AICD into U87 glioblastoma cells significantly suppressed LRP1 expression without affecting LDLR levels (see e.g., FIGS. 6A and 6B). The adaptor protein Fe65 has been previously shown to modulate AICD stability and potentiate its subsequent nuclear translocation (Cao and Sudhof, 2001). When overexpressed in U87 cells, Fe65 alone slightly suppressed LRP1 expression; however, co-expression of Fe65 with AICD further enhanced the AICD-mediated suppression of LRP1 expression (see e.g., FIGS. 6A and 6B). BACE1 cleavage of APP followed by γ-secretase cleavage generates A040 or A042 with concomitant production of AICD consisting of either 59 or 57 amino acids, respectively (referred to as AICD C57 and C59).

To test whether AICD can rescue LRP1 expression in cells deficient for either PS or APP, AICD C50, C57, and C59 were cloned into a retroviral vector. Infection with retrovirus expressing AICD C50, C57, or C59 significantly suppressed LRP1 expression in MEF cells deficient for either PS1/2 or APP/APLP2 (see e.g., FIGS. 6C, 6D, 6F, and 6G). A reduction in LRP1 mRNA levels was also observed when analyzed by real-time PCR (see e.g., FIGS. 6E and 6H). These data indicate that AICD can reduce the defective LRP1 expression observed in APP/APLP2-DKO and PS-DKO cells and demonstrate that an AICD-dependent signaling pathway is crucial for the regulation of LRP1 cellular levels.

### Example 8

### AICD Nuclear Signaling Inhibits LRP1 Promoter Activation

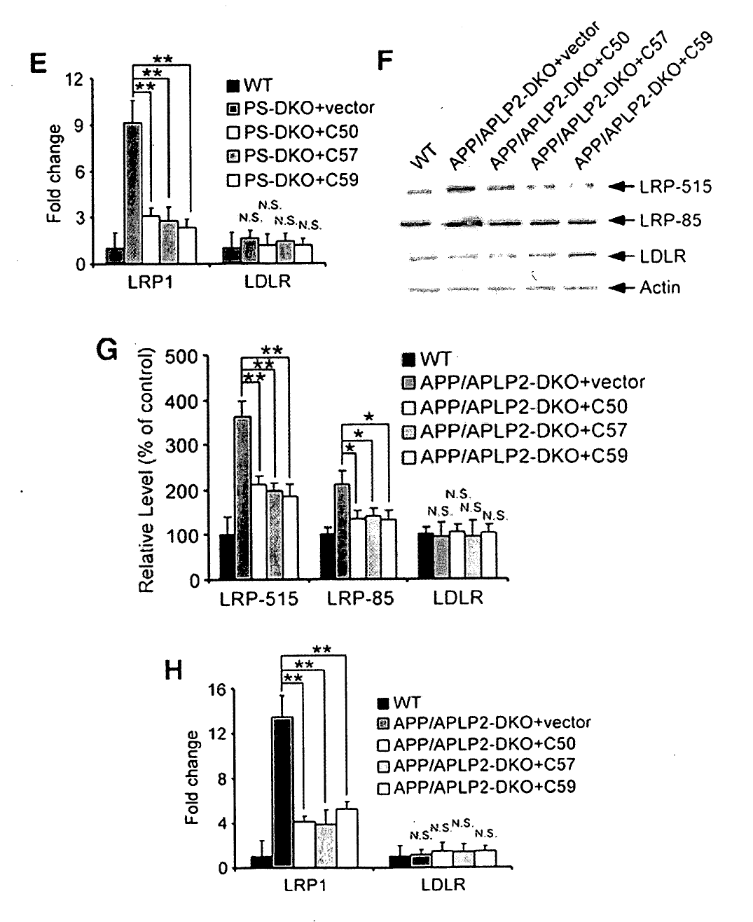

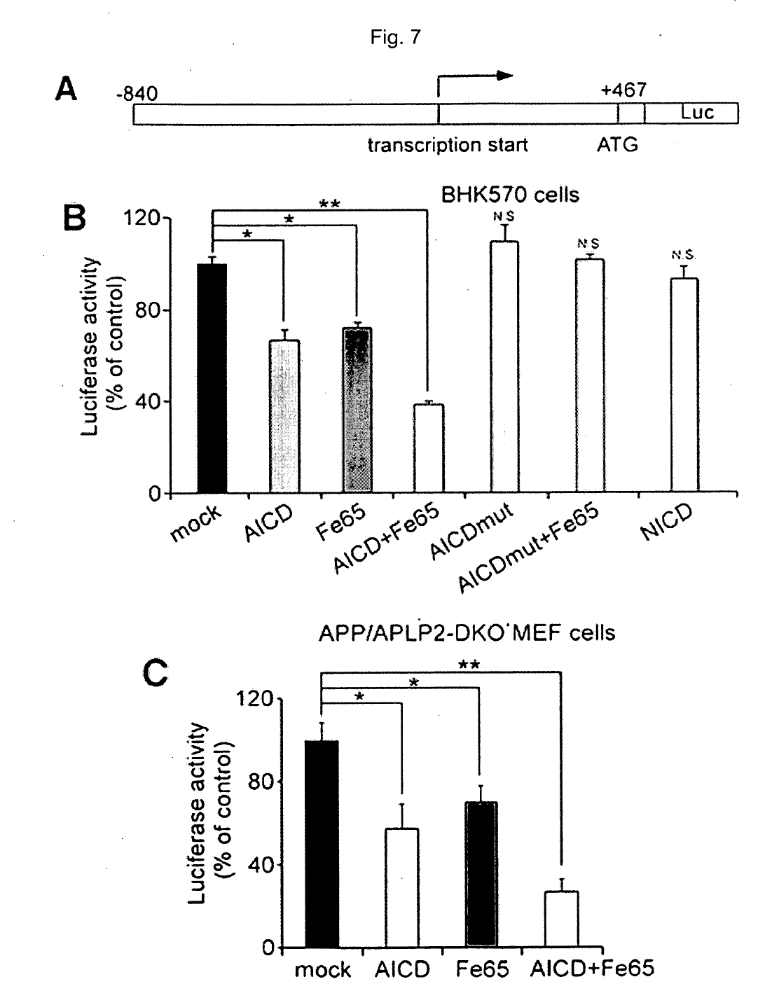

This example illustrates the results of experiments designed to test whether LRP1 promoter activity is repressed by AICD. The LRP1 promoter was cloned into a luciferase reporter vector pGL3 (see e.g., FIG. 7A), and its activity was measured in BHK570 cells and APP/APLP2-DKO MEF cells after transfection of AICD, Fe65, or both. Results show that AICD reduced LRP1 promoter activity and Fe65 further potentiated this effect in both cell types (see e.g., FIGS. 7B and 7C). An AICD mutant bearing a functional mutation in the 682YxNPxY motif (Y682G, see Borg et al., Mol. Cell. Biol. 16, 6229-6241, 1996) lost the ability to regulate LRP1 promoter, and Notch intracellular domain (NICD) did not change LRP1 promoter function in this assay (see e.g., FIG. 7B). To examine whether AICD binds directly to the LRP1 promoter, a chromatin immunoprecipitation (ChIP) assay was performed. Immunoprecipitation of mouse brain lysates with an antibody that recognizes the APP C-terminal domain showed that AICD associates with the LRP1 promoter (see e.g., FIGS. 7D and 7E). The ability of antibodies to Fe65 and Tip60 to immunoprecipitate the LRP1 promoter was also analyzed. It was found that both Fe65 and Tip60 antibodies immunoprecipitated the LRP1 promoter (see e.g., FIGS. 7D and 7E). The association of AICD, Fe65, and Tip60 with the LRP1 promoter was specific because normal rabbit IgG failed to immunoprecipitate the LRP1 promoter. In addition, APP, Fe65, and Tip60 antibodies did not precipitate a control Notch target promoter, HES1 (see e.g., FIG. 7D). Further, the association of Fe65 and Tip60 with LRP1 promoter was greatly reduced in APP-KO mouse brain (see e.g., FIG. 7D). These results show that AICD, together with Fe65 and Tip60, bind directly to the LRP1 promoter to suppress its activation.

### Example 9

### AICD Rescues ApoE and Cholesterol Defects in Cells Lacking APP/APLP2 or PS

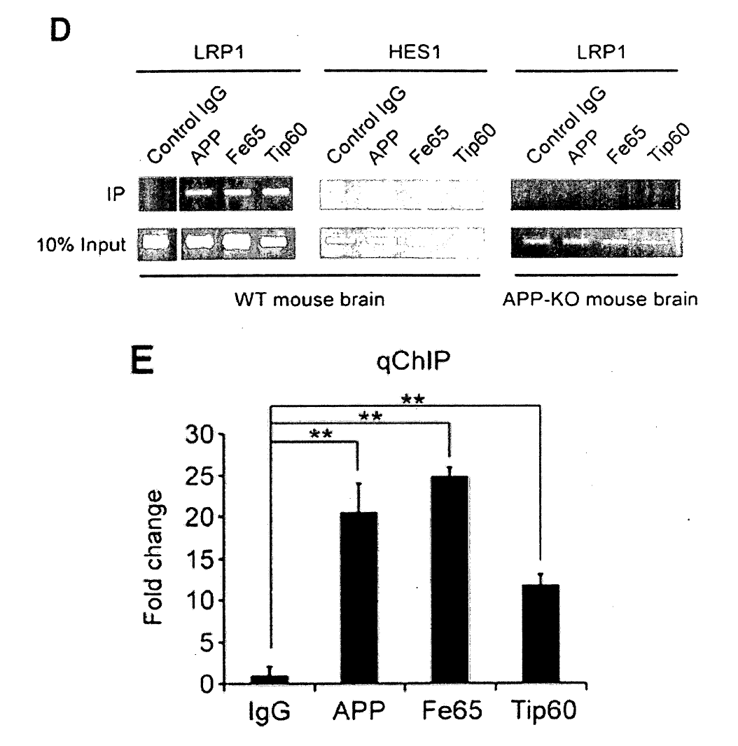

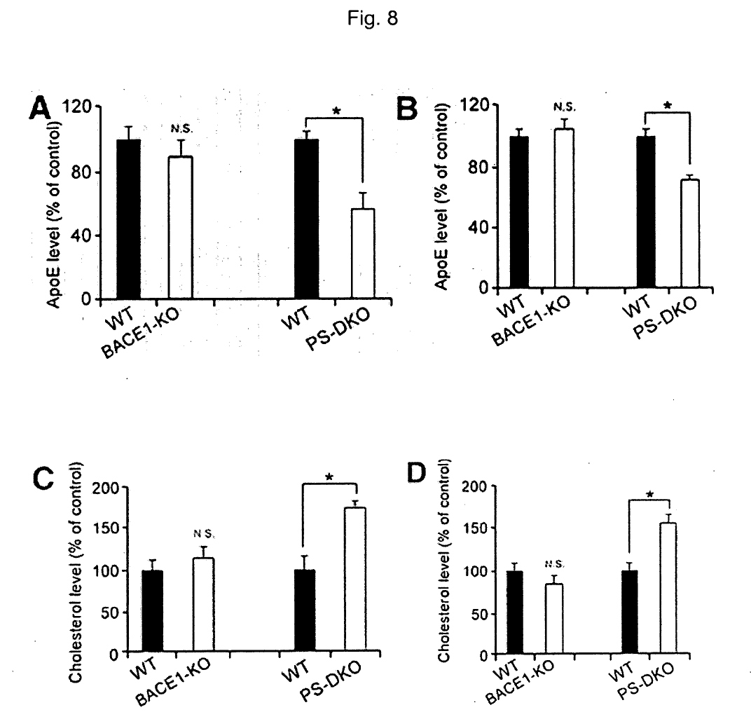

Having demonstrated that LRP1 is a major receptor that regulates apoE and cholesterol metabolism and that γ-secretase activity is required for APP/APLP2-mediated regulation of LRP1 expression, the potential alterations in apoE and cholesterol levels in PS-DKO MEF cells and mouse brain was investigated. Results show that apoE levels were decreased while cholesterol levels were increased in PS-DKO MEF cells (see e.g., FIGS. 8A and 8C) and mouse brain (see e.g., FIGS. 8B and 8D) when compared to their WT controls. Normal levels of apoE and cholesterol were found in BACE1-KO MEF cells and BACE1-KO mouse brain (see e.g., FIG. 8A-D), demonstrating that BACE1 is not involved in apoE and cholesterol metabolism. Since AICD rescued LRP1 expression in MEF cells devoid of either APP/APLP2 or PS, experiments were performed to analyze whether forced expression of AICD could restore apoE and cholesterol levels in these cells. Forced expression of AICD in APP/APLP2-DKO (see e.g., FIG. 8E) and PS-DKO (see e.g., FIG. 8F) MEF cells significantly increased apoE levels when compared to MEF cells infected with vector alone, albeit not to the levels observed in WT control cells. Likewise, AICD, but not vector alone, partially restored cholesterol levels in APP/APLP2-DKO (see e.g., FIG. 8G) and PSDKO (see e.g., FIG. 8H) MEF cells. These results confirmed that AICD-mediated control of LRP1 expression is a key regulatory pathway in brain apoE and cholesterol homeostasis.

### Example 10

### In Vivo Evidence that LRP1 is a Major Brain ApoE/Lipoprotein Receptor Critical for Spine/Synaptic Integrity and Function

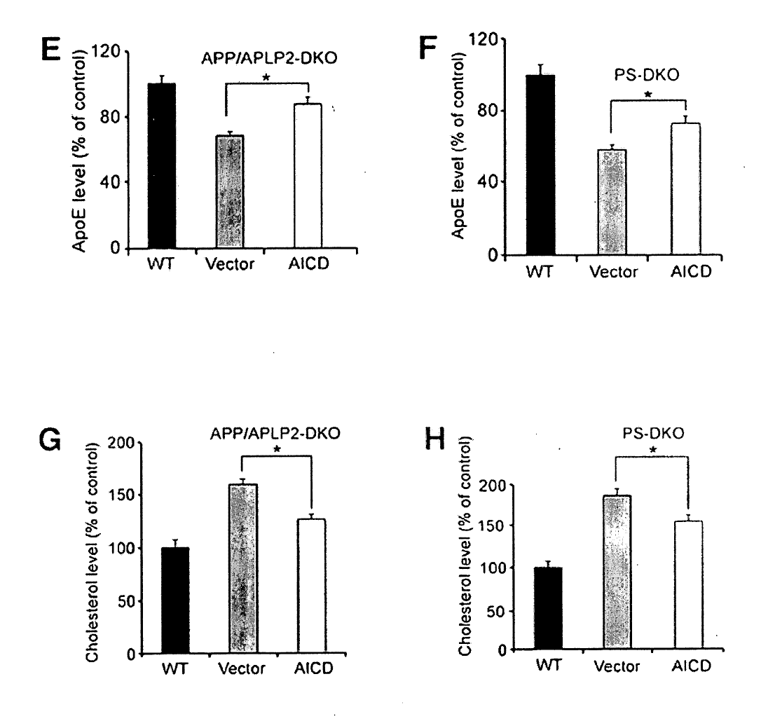

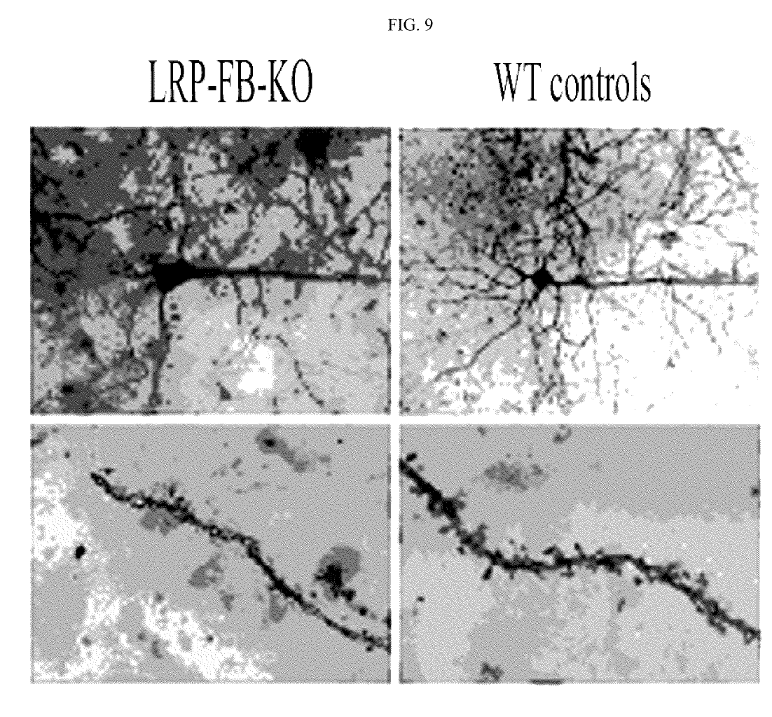

To further define the in vivo function of LRP in apoE/lipoprotein metabolism, we analyzed several brain lipids associated with apoE/lipoprotein in conditional LRP forebrain knockout mice (Liu et al., Neuron 56, 66-78, 2007). In addition to cholesterol, we found that several brain lipids (e.g., sulfatide and cerebroside) critical for spine/synaptic integrity and functions were also significantly decreased in LRP-FB-KO mice (data not shown). The reduced brain lipid levels prompted us to analyze neuronal spine and synaptic integrity and synaptic functions. Brains from LRP-FB-KO mice and WT controls at one year of age were subjected to comparative microscopic assessment of the Golgi-stained neurons. The WT cortical neurons are well branched and well spined, whereas cortical neurons from the LRP-FB-KO mice are less branched and poorly spined (see e.g., FIG. 9). These results show that there is significant loss of neuronal spines in LRP-FB-KO neurons compared to WT littermate controls.

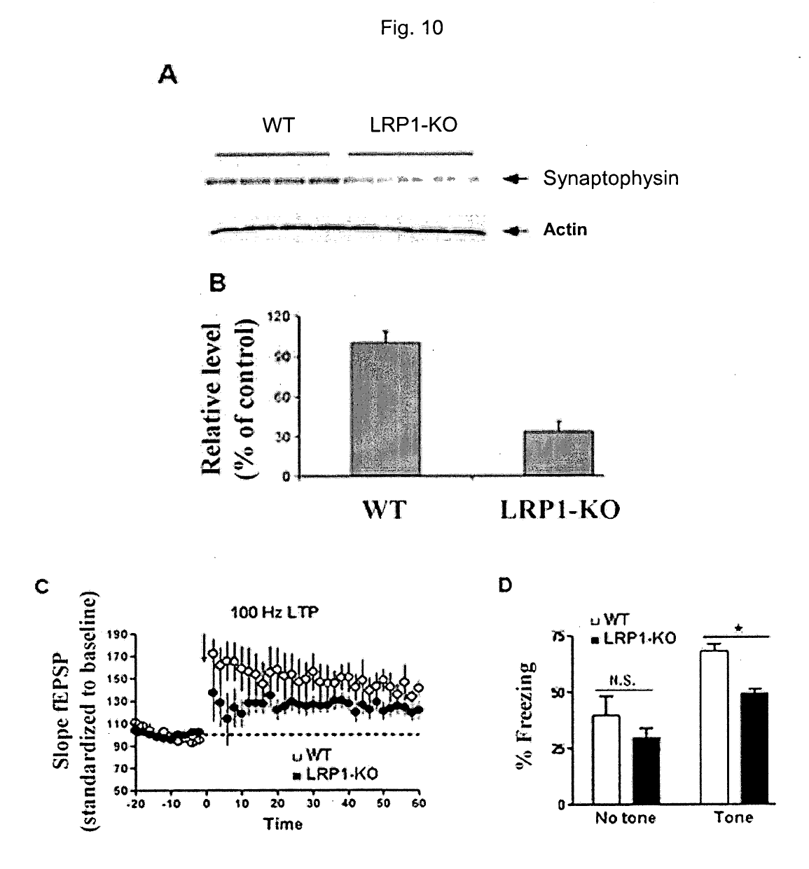

The levels of synaptophysin, a marker for neuronal synapses, were also reduced in LRP-FB-KO brains compared to WT littermate controls (see e.g., FIGS. 10A and 10B). LRP-FB-KO mice exhibited impaired long-term potentiation (LTP) (see e.g., FIG. 10C) and memory deficits as measured by fear-conditioning memory tests (see e.g., FIG. 10D). These results demonstrate that LRP expression is crucial for neuronal spine/synaptic integrity and functions, likely because of its important role in modulating brain apoE/lipid metabolism and/or signaling.

### Example 11

### LRP Expression is Critical for Neuronal Survival

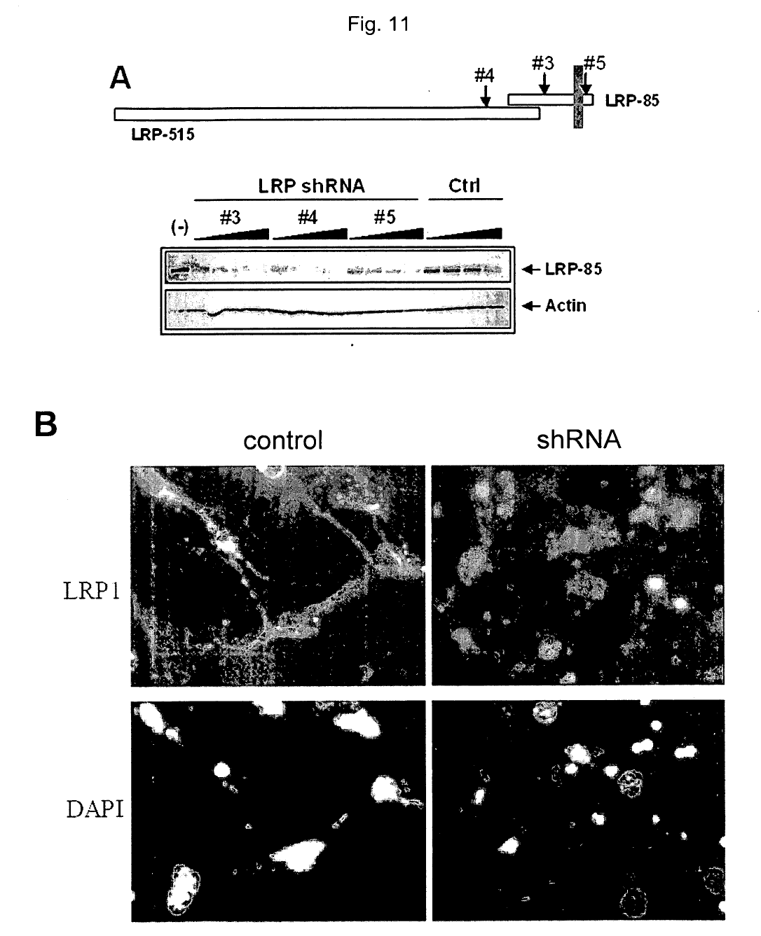

To further assess potential impacts of neuronal LRP loss-of-function, we examined effects of LRP knockdown in primary neurons on neuronal survival. Primary hippocampal neurons from E15 of WT C57BL/6 mice were isolated and cultured for 7 days. First, we tested five lentiviral shRNAs (Sigma Mission™) for their effectiveness in reducing LRP expression and found that three of these lentiviral shRNA were effective in knocking down LRP expression by >90% (see e.g., FIGS. 11A and 11B). Primary neurons were infected with increasing amounts of LRP shRNA lentivirus (with targeting sites indicated in the diagram) or control pLKO.1 lentivirus (−). LRP levels were analyzed by Western blot using an antibody against the 85-kDa subunit of LRP and actin was blotted as a loading control (see e.g., FIG. 11 Å). These results show a dose-dependent knockdown of LRP expression by LRP lentiviral shRNA. In addition, control or LRP shRNA lentivirus-infected neurons were evaluated by immunofluorescence using a LRP-specific antibody and DAPI as a counterstain (FIG. 11B). This decreased immunoreactivity in neuronal processes and soma confirms shRNA-directed knockdown of LRP in primary neurons.

To examine the effects of LRP knockdown on neuronal survival, we performed TUNEL staining on primary neurons infected with either control lentivirus or LRP shRNA lentivirus (see e.g., FIG. 11C). LRP knockdown increased-TUNEL-positive neurons (arrows in FIG. 11C). These results show that LRP knockdown results in a significant increase in TUNEL-positive neurons and that decreased LRP expression leads to increased neuronal apoptosis.

To examine whether this event was mediated by caspase-dependent pathway, we compared caspase-3 activation in control and LRP shRNA knockdown neurons. Primary neurons were infected with control or shRNA lentivirus and the levels of procaspase-3 and active caspase-3 were determined by Western blot (see e.g., FIG. 11D). As a positive control for caspase activation, 0.5 μM staurosporine treatment was used while actin levels were determined as loading controls. These results show that activated caspase-3 is significantly increased when LRP expression is knocked down by shRNA.

We also found that activated Akt and β-catenin levels were both significantly decreased in LRP shRNA-treated neurons, which shows that several signaling pathways are responsible for LRP-dependent neuronal survival. Together, these results demonstrate that LRP is important for neuronal survival. Without being limited by theory, we believe that loss of LRP expression/function in neurons due to shedding or aberrant trafficking results in a significant decrease in neuronal signaling, survival, and functions.

### Example 12

### LRP1 Knockdown in Cells In Vitro

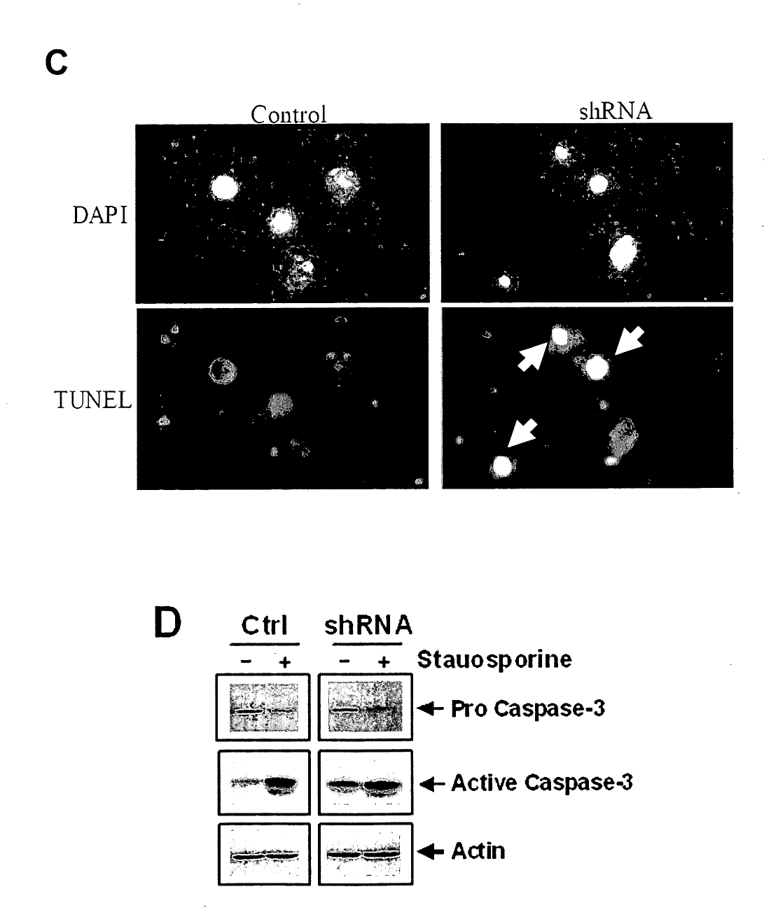

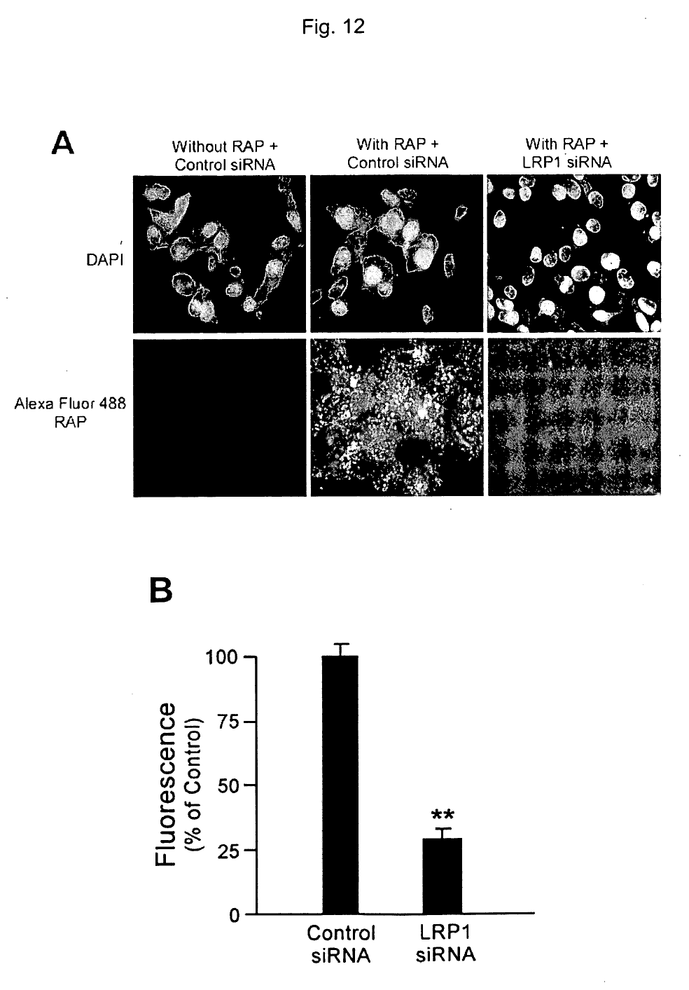

The present inventors have created a cell-based assay to measure fluorescently labeled LRP1 ligand uptake, in which ligand uptake correlates with LRP1 expression and transport function. Recent studies have shown that LRP1 expression can be inhibited by siRNA against LRP1 (Woodley, J. T. et al, J Cell Sci, 2009, 122:1495-1498). In this example, knockdown of LRP1 was assessed by measuring the amount of labeled Receptor-Associated Protein (RAP) labeled with Alexa Fluor488 that was associated with cells. In this example, Receptor-Associated Protein (RAP) labeled with Alexa Fluor488 was added to cell cultures comprising U87 glioblastoma cells, along with an siRNA against LRP1. Incubation of labeled RAP with cells for 10 min at 20 nM generated a good signal to noise ratio in control cultures. FIG. 12 portrays a representative assay in which LRP1 expression was knocked down by an LRP1-specific siRNA in U87 glioblastoma cells that normally express abundant LRP1. The data demonstrate that siRNA treatment can reduce cell-associated fluorescently labeled RAP by >70% (FIG. 12).

### Example 13

### LRP1 Overexpression can Lead to Enhanced Accumulation of Labelled LRP1 Ligand

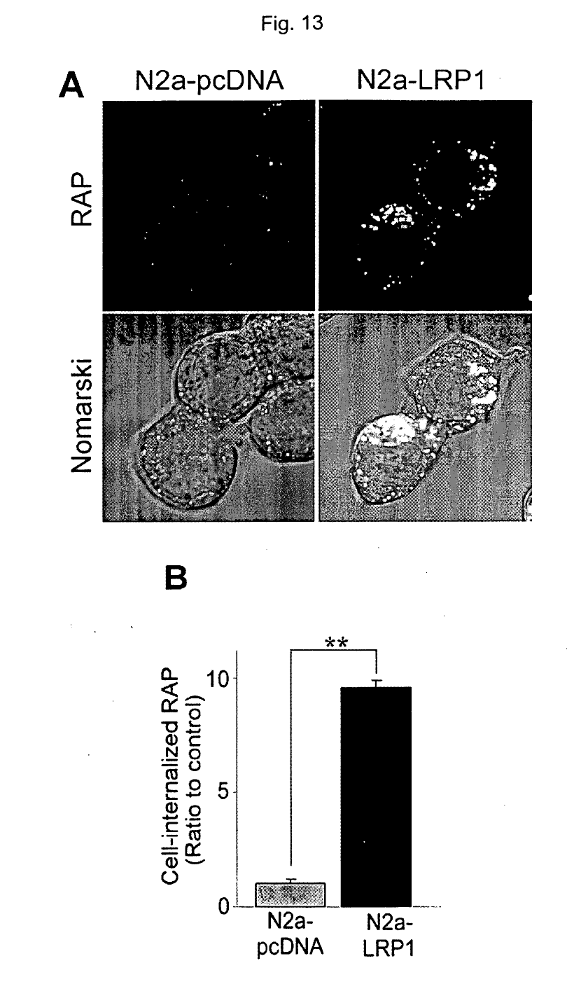

In these experiments, fluorescently labeled RAP (20 nM) was incubated with neuroblastoma N2a cells that had been stably transfected with either control vector (“pcDNA”) or with LRP1 cDNA (“LRP1”). Internalization of labeled RAP was assessed by both confocal microscopy (FIG. 13A) or fluorescently-activated cell sorting (FACS, FIG. 13B). The results demonstrates that, when LRP1 is overexpressed, cell associated signals for RAP are significantly increased, and provides proof-of-principle that increased LRP1 expression and/or transport function can be assayed.

### Example 14

### High Throughput Screening of Pharmacological Compounds that Upregulate LRP1 Expression and/or Transport Functions

The National Cancer Institute (NCI) Structural Diversity Set is screened for pharmacophores that upregulate LRP1 expression and/or transport function using high-throughput methods. The Structural Diversity Set consists of ˜1,990 compounds chosen from over 140,000 ‘Open’ compounds representing the unique array of pharmacophores present in the NCI repository. These compounds are distributed in the 10 center columns of 96-well plates (10×8 wells=80 wells), allowing the outside to be used for controls (total of 16 wells). The controls in quadruplicates are 1) empty wells without cells to determine the background binding to the plate; 2) cells without Alexa Fluor 488-RAP to determine auto fluorescence; 3) cells with 100 fold excess unlabeled RAP (2 μM) to determine non-specific uptake; and 4) with LRP1 knockdown to determine LRP1-independent RAP uptake.

Ten compounds that exhibit the greatest degree of reproducibly enhanced RAP uptake are selected for further testing to examine if these candidates regulate LRP1 expression and/or trafficking. These compounds are also validated in primary neurons to analyze their effectiveness in regulating LRP1 expression and/or transport function.

### Example 15

### Screening of Compounds In Vivo for Blood-Brain Barrier Permeability

In this Example, compounds identified in the high-throughput assay described in Example 14 are selected for in vivo testing. For the in vivo studies, LRP1-regulating compounds that cross the murine blood-brain barrier (BBB) are identified using published methods (Pan et al, 2004. J Cell Sci 117:5071-5078).

### Example 16

### Testing of Compounds for Neurotoxicity

In this Example, LRP1-regulating compounds, BBB-crossing compounds identified in the previous Examples are tested for their effect on AD pathology in two AD mouse models. Compounds identified by the above screening methods that significantly increase LRP1 expression are tested for neurotoxicity as measured by neuronal viability using a routine method well known to skilled artisans.

### Example 17

### Testing of Compounds for Bioavailability

Compounds having acceptable neurotoxicity as determined in the previous example are assessed for bioavailability, dosage, delivery method. These assessments are performed using pharmacokinetic-pharmacodynamic (PK-PD) modeling (e.g., Sheiner, L. B., et al., Ann. Rev. Pharmacol. Toxicol 40: 67-95, 2000) in WT C56BL/6 mice to determine an optimal dosage and delivery method and frequency.

### Example 18

### Testing of Compounds for Efficacy In Vivo

In these tests, apoE4-TR mice at 3 months of age are treated with a compound identified by the above methods for a total of three months, and the efficacy of each compound in upregulating LRP1 expression or activity in the brain is determined. LRP1 expression is assessed by Western blot to examine the total level of LRP1, and by immunofluorescence staining to determine specificity of expression pattern. In experiments with compounds that significantly increase LRP1 expression, the compound is further tested in vivo for enhancement of brain lipid metabolism, dendritic/synaptic integrity and memory performance.

### Example 19

### Testing of Compounds In Vivo for Effects on AD Pathology

APP/PS1 mice, which express a chimeric mouse/human APP containing the K595N/M596L Swedish mutations and a mutant human PS1 carrying the exon 9-deleted variant under the control of mouse prion promoter elements, are a model for AD. The mice develop amyloid plaques at 5-6 months of age. In order to test the effects of a compound identified in the above assays on AD pathology, APP/PS1 mice are treated with a selected compound starting at 3 months of age, for a total of three months. The effects of the compound on LRP1 expression, Aβ accumulation, amyloid plaque formation and memory performance are then assessed.

What is claimed is:

1. A method of identifying a compound for treating Alzheimer's disease, the method comprising:
a) forming an in vitro mixture comprising i) cells expressing LDL-receptor-related protein 1 (LRP1), ii) an LRP1 ligand comprising a label, and iii) a candidate compound; and
b) determining quantity of the label incorporated by the cells,
whereby the candidate compound is effective for treating Alzheimer's disease if the quantity of label incorporated by the cells exceeds that of a control in vitro mixture comprising cells expressing LRP1 and the LRP1 ligand, but not comprising the compound.

2. A method according to claim 1, wherein the cells expressing LRP1 are selected from the group consisting of hepatocytes, keratinocytes, embryonic cells, fibroblasts, macrophagess, Mueller glial cells, mammary epithelial cells, glioblastoma cells, retinal pigment epithelial cells, neurons, astrocytes, microglia, smooth muscle cells, endothelial cells and osteoblasts.

3. A method according to claim 1, wherein the cells expressing LRP1 are selected from the group consisting of keratinocytes and glioblastoma cells.

4. A method according to claim 1, wherein the cells expressing LRP1 are selected from the group consisting of human cells, mouse cells, rat cells, chicken cells, Xenopus cells, zebrafish cells, Drosophila cells and rabbit cells.

5. A method according to claim 1, wherein the cells expressing LRP1 are selected from the group consisting of human cells and mouse cells.

6. A method according to claim 1, wherein the cells expressing LRP1 are U87 glioblastoma cells.

7. A method according to claim 1, wherein the LRP1 ligand is selected from the group consisting of an anti-LRP1 antibody, apoE, lipoprotein lipase, hepatic lipase, tPA, uPA, factor IXa, Factor VIIIa, factor VIIa, TFPI, matrix metalloproteinase-13 (MMP-13), MMP-9, spingolipid activator protein (SAP), pregnancy zone protein, alpha2-macroglobulin, complement C3, PAI-1, C1 inhibitor, anitthrombin III, heparin cofactor II, alpha1-antitrypsin, APP, thrombospondin-1, thrombospondin-2, Pseudomonas exotoxin A, rhinovirus, receptor-associated protein (RAP), lactoferrin, heat-shock protein 96 (HSP96), HSP90alpha and HIV-Tat protein.

8. A method according to claim 7, wherein the LRP1 ligand is RAP.

9. A method according to claim 7, wherein the LRP1 ligand is an anti-LRP1 antibody.

10. A method according to claim 1, wherein the label is selected from the group consisting of an enzyme, a radioisotope, a fluorogen, fluorophore, a chromogen and a chromophore.

11. A method according to claim 10, wherein the enzyme is selected from the group consisting of a peroxidase, a phosphatase, a galactosidase and a luciferase.

12. A method according to claim 10, wherein the radioisotope is selected from the group consisting of a ³²P, a ³³P, ³⁵S, a ¹⁴C, an ¹²⁵I, an ¹³¹I and a ³H.

13. A method according to claim 10, wherein the fluorophore is selected from the group consisting of a fluorescein, a rhodamine, an Alexa Fluor®, an IRDye®, a coumarin, an indocyanine and a quantum dot.

14. A method according to claim 10, wherein the label is selected from the group consisting of a biotin, a digoxygenin, and a peptide comprising an epitope.

15. A method according to claim 10, wherein the fluorophore is AlexaFluor488®.


SEQUENCE LISTING (6 sequences)

SEQ ID NO: 1 - DNA, 18 bp, Artificial Sequence
  PCR primer
tcgggtgtccctgtttac

SEQ ID NO: 2 - DNA, 18 bp, Artificial Sequence
  PCR primer
gaaagcggtccaagagtg

SEQ ID NO: 3 - DNA, 20 bp, Artificial Sequence
  RT PCR primer
gggagcctgaaatcctagag

SEQ ID NO: 4 - DNA, 19 bp, Artificial Sequence
  RT PCR primer
ggaaagcggtccaagagtg

SEQ ID NO: 5 - DNA, 20 bp, Artificial Sequence
  PCR primer
cgtgtctcttcctcccattg

SEQ ID NO: 6 - DNA, 20 bp, Artificial Sequence
  PCR primer
gatccagtgtgatccgcagg